(19) United States
(12) Patent Application Publication
(10) Pub. No.: US 2023/0230655 A1
(43) Pub. Date: Jul. 20, 2023

(54) METHODS AND SYSTEMS FOR ASSESSING FIBROTIC DISEASE WITH DEEP LEARNING

(71) Applicant: CEDARS-SINAI MEDICAL CENTER, LOS ANGELES, CA (US)

(72) Inventors: Dermot P. McGovern, Los Angeles, CA (US); Dalin Li, Walnut, CA (US)

(21) Appl. No.: 18/050,837

(22) Filed: Oct. 28, 2022

Related U.S. Application Data
(63) Continuation of application No. PCT/US2021/029962, filed on Apr. 29, 2021.
(60) Provisional application No. 63/018,377, filed on Apr. 30, 2020.

(51) Int. Cl. G16B 20/00; G16B 30/00; G16B 40/20; G16H 50/20

(52) U.S. Cl. CPC G16B 20/00; G16B 30/00; G16B 40/20; G16H 50/20

(57) ABSTRACT

The present disclosure provides methods and systems of identifying a fibrotic disease in a subject using a DeepLearning model. The DeepLearning model may be used to predict, treat, monitor, and/or prevent the fibrotic disease in the subject, as well as to characterize a subtype of the fibrotic disease.

### CROSS-REFERENCE

This application is a continuation of International Application No. PCT/US2021/029962 filed Apr. 29, 2021 which claims the benefit of U.S. Provisional Patent Application No. 63/018,377, filed Apr. 30, 2020, each of which is incorporated by reference herein in its entirety.

### STATEMENT AS TO FEDERALLY SPONSORED RESEARCH

This invention was made with government support under Grant No. DK108140, DK046763, DK062413, DK046763, HS021747, and AI067068, awarded by the National Institutes of Health. The government has certain rights in the invention.

### BACKGROUND

Fibrotic diseases and disorders may have a significant effect on morbidity and quality of life. Fibrotic diseases and disorders affect millions of people in the United States. The significant effect on morbidity is, in part, due to limitations of existing diagnostic and prognostic tests that fail to identify patients suffering from fibrotic diseases early enough in disease progression to prevent worsening of the disease or development of complications, such as certain severe or advanced-stage disease phenotypes.

### SUMMARY

Provided herein are methods and systems for assessing non-inflammatory diseases or conditions (e.g., fibrotic diseases) in subjects using DeepLearning (DL) prediction models. The DL prediction models are applied to a non-inflammatory disease (e.g., fibrotic disease) profile of a biological sample of a subject to identify a presence or an absence of the non-inflammatory disease or condition (e.g., fibrotic disease) in the subject, or a likelihood that the subject will develop the non-inflammatory disease or condition (e.g., fibrotic disease). The non-inflammatory disease (e.g., fibrotic disease) profile may comprise quantitative measures of a plurality of genomic loci containing, for example, genetic variants that are associated with the non-inflammatory disease or condition (e.g., fibrotic disease).

Aspects disclosed herein provide methods for identifying an non-inflammatory disease or condition in a subject, comprising: (a) assaying a biological sample of the subject to generate a dataset comprising genetic data; (b) processing the dataset at a plurality of genomic loci to determine quantitative measures of each genomic locus of the plurality of genomic loci, wherein the plurality of genomic loci comprises non-inflammatory disease-associated genes, thereby producing an non-inflammatory disease profile of the biological sample of the subject; and (c) applying a deep learning prediction model to the non-inflammatory disease profile to identify a presence of the non-inflammatory disease or condition in the subject, or a likelihood that the subject will develop the non-inflammatory disease or condition. In some embodiments, the non-inflammatory disease or condition comprises cardiovascular disease, adolescent idiopathic scoliosis, diabetes, a neurological disease, a fibrotic disease, or obesity. In some embodiments, the fibrotic disease comprises Primary Sclerosing Cholangitis (PSC), scleroderma, or pulmonary fibrosis. In some embodiments, the fibrotic disease comprises the PSC. In some embodiments, the fibrotic disease comprises the scleroderma. In some embodiments, the fibrotic disease comprises the pulmonary fibrosis. In some embodiments, the diabetes comprises type 2 diabetes. In some embodiments, the neurological disease comprises Alzheimer's disease. In some embodiments, the biological sample is selected from the group consisting of: a whole blood sample, a deoxyribonucleic acid (DNA) sample, a ribonucleic acid (RNA) sample, a cell-free sample, a tissue sample, a cell sample, and a derivative or fraction thereof. In some embodiments, assaying the biological sample comprises sequencing the biological sample to generate the dataset. In some embodiments, the method further comprises identifying the presence of the non-inflammatory disease or condition in the subject, or the likelihood that the subject will develop the non-inflammatory disease or condition, at a sensitivity of at least about 70%. In some embodiments, the method further comprises identifying the presence of the non-inflammatory disease or condition in the subject, or the likelihood that the subject will develop the non-inflammatory disease or condition, at a sensitivity of at least about 80%. In some embodiments, the method further comprises identifying the presence of the non-inflammatory disease or condition in the subject, or the likelihood that the subject will develop the non-inflammatory disease or condition, at a sensitivity of at least about 90%. In some embodiments, the method further comprises identifying the presence of the non-inflammatory disease or condition in the subject, or the likelihood that the subject will develop the non-inflammatory disease or condition, at a sensitivity of at least about 95%. In some embodiments, the method further comprises identifying the presence of the non-inflammatory disease or condition in the subject, or the likelihood that the subject will develop the non-inflammatory disease or condition, at a sensitivity of at least about 99%. In some embodiments, the method further comprises identifying the presence of the non-inflammatory disease or condition in the subject, or the likelihood that the subject will develop the non-inflammatory disease or condition, at a specificity of at least about 70%. In some embodiments, the method further comprises identifying the presence of the non-inflammatory disease or condition in the subject, or the likelihood that the subject will develop the non-inflammatory disease or condition, at a specificity of at least about 80%. In some embodiments, the method further comprises identifying the presence of the non-inflammatory disease or condition in the subject, or the likelihood that the subject will develop the non-inflammatory disease or condition, at a specificity of at least about 90%. In some embodiments, the method further comprises identifying the presence of the non-inflammatory disease or condition in the subject, or the likelihood that the subject will develop the non-inflammatory disease or condition, at a specificity of at least about 95%. In some embodiments, the method further comprises identifying the presence of the non-inflammatory disease or condition in the subject, or the likelihood that the subject will develop the non-inflammatory disease or condition, at a specificity of at least about 99%. In some embodiments, the method further comprises identifying the presence of the non-inflammatory disease or condition in the subject, or the likelihood that the subject will develop the non-inflammatory disease or condition, at a positive predictive value (PPV) of at least about 70%. In some embodiments, the method further comprises identifying the presence of the non-inflammatory disease or condition in the subject, or the likelihood that the subject will develop the non-inflammatory disease or condition, at a PPV of at least about 80%. In some embodiments, the method further comprises identifying the presence of the non-inflammatory disease or condition in the subject, or the likelihood that the subject will develop the non-inflammatory disease or condition, at a PPV of at least about 90%. In some embodiments, the method further comprises identifying the presence of the non-inflammatory disease or condition in the subject, or the likelihood that the subject will develop the non-inflammatory disease or condition, at a PPV of at least about 95%. In some embodiments, the method further comprises identifying the presence of the non-inflammatory disease or condition in the subject, or the likelihood that the subject will develop the non-inflammatory disease or condition, at a positive PPV of at least about 99%. In some embodiments, the method further comprises identifying the presence of the non-inflammatory disease or condition in the subject, or the likelihood that the subject will develop the non-inflammatory disease or condition, at a negative predictive value (NPV) of at least about 70%. In some embodiments, the method further comprises identifying the presence of the non-inflammatory disease or condition in the subject, or the likelihood that the subject will develop the non-inflammatory disease or condition, at a NPV of at least about 80%. In some embodiments, the method further comprises identifying the presence of the non-inflammatory disease or condition in the subject, or the likelihood that the subject will develop the non-inflammatory disease or condition, at a NPV of at least about 90%. In some embodiments, the method further comprises identifying the presence of the non-inflammatory disease or condition in the subject, or the likelihood that the subject will develop the non-inflammatory disease or condition, at a NPV of at least about 95%. In some embodiments, the method further comprises identifying the presence of the non-inflammatory disease or condition in the subject, or the likelihood that the subject will develop the non-inflammatory disease or condition, at a NPV of at least about 99%. In some embodiments, the method further comprises identifying the presence of the non-inflammatory disease or condition in the subject, or the likelihood that the subject will develop the non-inflammatory disease or condition, with an Area Under Curve (AUC) of at least about 0.70. In some embodiments, the method further comprises identifying the presence of the non-inflammatory disease or condition in the subject, or the likelihood that the subject will develop the non-inflammatory disease or condition, with an AUC of at least about 0.80. In some embodiments, the method further comprises identifying the presence of the non-inflammatory disease or condition in the subject, or the likelihood that the subject will develop the non-inflammatory disease or condition, with an AUC of at least about 0.90. In some embodiments, the method further comprises identifying the presence of the non-inflammatory disease or condition in the subject, or the likelihood that the subject will develop the non-inflammatory disease or condition, with an AUC of at least about 0.95. In some embodiments, the method further comprises identifying the presence of the non-inflammatory disease or condition in the subject, or the likelihood that the subject will develop the non-inflammatory disease or condition, with an AUC of at least about 0.99. In some embodiments, the subject is asymptomatic for one or more non-inflammatory disease or conditions. In some embodiments, the deep learning prediction model is trained using a first set of independent training samples associated with a presence of the non-inflammatory disease or condition and a second set of independent training samples associated with an absence of the non-inflammatory disease or condition. In some embodiments, the method further comprises applying the deep learning prediction model (e.g., a deep learning classifier) to a set of clinical health data of the subject. In some embodiments, the set of clinical health data comprises one or more of familial history of an non-inflammatory disease or disorder, age, hypertension or pre-hypertension, diabetes or pre-diabetes, overweight or obesity, environmental exposure, lifestyle risk factors (e.g., smoking, alcohol consumption, or drug use), or presence of other risk factors. In some embodiments, the deep learning prediction model comprises a deep learning algorithm, a neural network, a Random Forest, an XGBoost, a Gradient Boost, or a combination thereof. In some embodiments, the deep learning prediction model comprises a deep learning algorithm. In some embodiments, the deep learning algorithm comprises a deep neural network. In some embodiments, the deep neural network comprises a convolutional neural network (CNN). In some embodiments, the method further comprises optimizing a set of hyperparameters of the CNN. In some embodiments, optimizing the set of hyperparameters comprises performing an intensive grid search. In some embodiments, the set of hyperparameters comprises a number of layers and/or a number of neurons of the CNN. In some embodiments, the CNN comprises a combination of a plurality of CNNs. In some embodiments, the plurality of CNNs comprises two CNNs. In some embodiments, (a) comprises (i) subjecting the biological sample to conditions that are sufficient to isolate, enrich, or extract a plurality of DNA molecules; and (ii) analyzing the plurality of DNA molecules to generate the dataset. In some embodiments, the plurality of genomic loci comprises at least about 1,000 distinct genomic loci. In some embodiments, the plurality of genomic loci comprises at least about 10,000 distinct genomic loci. In some embodiments, the plurality of genomic loci comprises at least about 100,000 distinct genomic loci. In some embodiments, the method further comprises identifying the likelihood that the subject will develop the non-inflammatory disease or condition. In some embodiments, the method further comprises providing a therapeutic intervention for the non-inflammatory disease or condition of the subject, provided the presence of the non-inflammatory disease or condition is identified in the subject. In some embodiments, the method further comprises monitoring the non-inflammatory disease or condition of the subject by assessing the non-inflammatory disease or condition in the subject at a plurality of time points, wherein the assessing is based at least partially on identifying the presence of the non-inflammatory disease or condition in (c) at one or more time points of the plurality of time points. In some embodiments, a difference between two or more assessments of the non-inflammatory disease or condition in the subject at two or more time points of the plurality of time points is indicative of one or more of: (i) a diagnosis of the non-inflammatory disease or condition of the subject, (ii) a prognosis of the non-inflammatory disease or condition of the subject, or (iii) an efficacy or non-efficacy of a course of treatment for treating the non-inflammatory disease or condition of the subject.

In another aspect, the present disclosure provides a method for identifying a fibrotic disease in a subject, comprising: (a) assaying a biological sample of the subject to generate a dataset comprising genetic data; (b) processing the dataset at a plurality of genomic loci to determine quantitative measures of each genomic locus of the plurality of genomic loci, wherein the plurality of genomic loci comprises fibrotic disease-associated genes, thereby producing fibrotic disease profile of the biological sample of the subject; and (c) applying a deep learning prediction model to the fibrotic disease profile to identify a presence or an absence of the fibrotic disease in the subject, or a likelihood that the subject will develop the fibrotic disease. In some embodiments, the fibrotic disease comprises Primary Sclerosing Cholangitis (PSC), scleroderma, or pulmonary fibrosis. In some embodiments, the fibrotic disease comprises the PSC. In some embodiments, the fibrotic disease comprises the scleroderma. In some embodiments, the fibrotic disease comprises the pulmonary fibrosis. In some embodiments, the biological sample is selected from the group consisting of: a whole blood sample, a deoxyribonucleic acid (DNA) sample, a ribonucleic acid (RNA) sample, a cell-free sample, a tissue sample, a cell sample, and a derivative or fraction thereof. In some embodiments, assaying the biological sample comprises sequencing the biological sample to generate the dataset. In some embodiments, the method further comprises identifying the presence or the absence of the fibrotic disease in the subject, or the likelihood that the subject will develop the fibrotic disease, at a sensitivity of at least about 70%. In some embodiments, the method further comprises identifying the presence or the absence of the fibrotic disease in the subject, or the likelihood that the subject will develop the fibrotic disease, at a sensitivity of at least about 80%. In some embodiments, the method further comprises identifying the presence or the absence of the fibrotic disease in the subject, or the likelihood that the subject will develop the fibrotic disease, at a sensitivity of at least about 90%. In some embodiments, the method further comprises identifying the presence or the absence of the fibrotic disease in the subject, or the likelihood that the subject will develop the fibrotic disease, at a sensitivity of at least about 95%. In some embodiments, the method further comprises identifying the presence or the absence of the fibrotic disease in the subject, or the likelihood that the subject will develop the fibrotic disease, at a sensitivity of at least about 99%. In some embodiments, the method further comprises identifying the presence or the absence of the fibrotic disease in the subject, or the likelihood that the subject will develop the fibrotic disease, at a specificity of at least about 70%. In some embodiments, the method further comprises identifying the presence or the absence of the fibrotic disease in the subject, or the likelihood that the subject will develop the fibrotic disease, at a specificity of at least about 80%. In some embodiments, the method further comprises identifying the presence or the absence of the fibrotic disease in the subject, or the likelihood that the subject will develop the fibrotic disease, at a specificity of at least about 90%. In some embodiments, the method further comprises identifying the presence or the absence of the fibrotic disease in the subject, or the likelihood that the subject will develop the fibrotic disease, at a specificity of at least about 95%. In some embodiments, the method further comprises identifying the presence or the absence of the fibrotic disease in the subject, or the likelihood that the subject will develop the fibrotic disease, at a specificity of at least about 99%. In some embodiments, the method further comprises identifying the presence or the absence of the fibrotic disease in the subject, or the likelihood that the subject will develop the fibrotic disease, at a positive predictive value (PPV) of at least about 70%. In some embodiments, the method further comprises identifying the presence or the absence of the fibrotic disease in the subject, or the likelihood that the subject will develop the fibrotic disease, at a PPV of at least about 80%. In some embodiments, the method further comprises identifying the presence or the absence of the fibrotic disease in the subject, or the likelihood that the subject will develop the fibrotic disease, at a PPV of at least about 90%. In some embodiments, the method further comprises identifying the presence or the absence of the fibrotic disease in the subject, or the likelihood that the subject will develop the fibrotic disease, at a PPV of at least about 95%. In some embodiments, the method further comprises identifying the presence or the absence of the fibrotic disease in the subject, or the likelihood that the subject will develop the fibrotic disease, at a positive PPV of at least about 99%. In some embodiments, the method further comprises identifying the presence or the absence of the fibrotic disease in the subject, or the likelihood that the subject will develop the fibrotic disease, at a negative predictive value (NPV) of at least about 70%. In some embodiments, the method further comprises identifying the presence or the absence of the fibrotic disease in the subject, or the likelihood that the subject will develop the fibrotic disease, at a NPV of at least about 80%. In some embodiments, the method further comprises identifying the presence or the absence of the fibrotic disease in the subject, or the likelihood that the subject will develop the fibrotic disease, at a NPV of at least about 90%. In some embodiments, the method further comprises identifying the presence or the absence of the fibrotic disease in the subject, or the likelihood that the subject will develop the fibrotic disease, at a NPV of at least about 95%. In some embodiments, the method further comprises identifying the presence or the absence of the fibrotic disease in the subject, or the likelihood that the subject will develop the fibrotic disease, at a NPV of at least about 99%. In some embodiments, the method further comprises identifying the presence or the absence of the fibrotic disease in the subject, or the likelihood that the subject will develop the fibrotic disease, with an Area Under Curve (AUC) of at least about 0.70. In some embodiments, the method further comprises identifying the presence or the absence of the fibrotic disease in the subject, or the likelihood that the subject will develop the fibrotic disease, with an Area Under Curve (AUC) of at least about 0.80. In some embodiments, the method further comprises identifying the presence or the absence of the fibrotic disease in the subject, or the likelihood that the subject will develop the fibrotic disease, with an Area Under Curve (AUC) of at least about 0.90. In some embodiments, the method further comprises identifying the presence or the absence of the fibrotic disease in the subject, or the likelihood that the subject will develop the fibrotic disease, with an Area Under Curve (AUC) of at least about 0.95. In some embodiments, the method further comprises identifying the presence or the absence of the fibrotic disease in the subject, or the likelihood that the subject will develop the fibrotic disease, with an Area Under Curve (AUC) of at least about 0.99. In some embodiments, the subject is asymptomatic for one or more fibrotic diseases. In some embodiments, the deep learning prediction model is trained using a first set of independent training samples associated with a presence of the non fibrotic disease and a second set of independent training samples associated with an absence of the fibrotic disease. In some embodiments, the method further comprises applying the deep learning prediction model (e.g., a deep learning classifier) to a set of clinical health data of the subject. In some embodiments, the set of clinical health data comprises one or more of familial history of a fibrotic disease, age, hypertension or pre-hypertension, diabetes or pre-diabetes, overweight or obesity, environmental exposure, lifestyle risk factors (e.g., smoking, alcohol consumption, or drug use), or presence of other risk factors. In some embodiments, the deep learning prediction model comprises a deep learning algorithm, a neural network, a Random Forest, an XGBoost, a Gradient Boost, or a combination thereof. In some embodiments, the deep learning prediction model comprises a deep learning algorithm. In some embodiments, the deep learning algorithm comprises a deep neural network. In some embodiments, the deep neural network comprises a convolutional neural network (CNN). In some embodiments, the method further comprises optimizing a set of hyperparameters of the CNN. In some embodiments, optimizing the set of hyperparameters comprises performing an intensive grid search. In some embodiments, the set of hyperparameters comprises a number of layers and/or a number of neurons of the CNN. In some embodiments, the CNN comprises a combination of a plurality of CNNs. In some embodiments, the plurality of CNNs comprises two CNNs. In some embodiments, (a) comprises (i) subjecting the biological sample to conditions that are sufficient to isolate, enrich, or extract a plurality of DNA molecules; and (ii) analyzing the plurality of DNA molecules to generate the dataset. In some embodiments, the plurality of genomic loci comprises at least about 1,000 distinct genomic loci. In some embodiments, the plurality of genomic loci comprises at least about 10,000 distinct genomic loci. In some embodiments, the plurality of genomic loci comprises at least about 100,000 distinct genomic loci. In some embodiments, the method further comprises identifying the likelihood that the subject will develop the fibrotic disease. In some embodiments, the method further comprises providing a therapeutic intervention for the fibrotic disease of the subject, provided the presence of the fibrotic disease is identified in the subject. In some embodiments, the method further comprises monitoring the fibrotic disease of the subject by assessing the fibrotic disease in the subject at a plurality of time points, wherein the assessing is based at least partially on identifying the presence or the absence of the fibrotic disease in (c) at one or more time points of the plurality of time points. In some embodiments, a difference between two or more assessments of the fibrotic disease in the subject at two or more time points of the plurality of time points is indicative of one or more of: (i) a diagnosis of the fibrotic disease of the subject, (ii) a prognosis of the fibrotic disease of the subject, or (iii) an efficacy or non-efficacy of a course of treatment for treating the fibrotic disease of the subject.

Aspects disclosed herein provide computer systems for identifying an non-inflammatory disease in a subject, comprising: (a) a database that is configured to store a dataset comprising genetic data, wherein the genetic data is obtained by assaying a biological sample of the subject; and (b) one or more computer processors operatively coupled to the database, wherein the one or more computer processors are individually or collectively programmed to: (i) process the dataset at a plurality of genomic loci to determine quantitative measures of each genomic locus of the plurality of genomic loci, wherein the plurality of genomic loci comprises non-inflammatory disease-associated genes, thereby producing an non-inflammatory disease profile of the biological sample of the subject; and (ii) apply a deep learning prediction model to the non-inflammatory disease profile to identify a presence of the non-inflammatory disease or condition in the subject, or a likelihood that the subject will develop the non-inflammatory disease or condition. In some embodiments, the non-inflammatory disease or condition comprises cardiovascular disease, adolescent idiopathic scoliosis, diabetes, a neurological disease, a fibrotic disease, or obesity. In some embodiments, the fibrotic disease comprises Primary Sclerosing Cholangitis (PSC), scleroderma, or pulmonary fibrosis. In some embodiments, the fibrotic disease comprises the PSC. In some embodiments, the fibrotic disease comprises the scleroderma. In some embodiments, the fibrotic disease comprises the pulmonary fibrosis. In some embodiments, the diabetes comprises type 2 diabetes. In some embodiments, the neurological disease comprises Alzheimer's disease. In some embodiments, the biological sample is selected from the group consisting of: a whole blood sample, a DNA sample, a RNA sample, a cell-free sample, a tissue sample, a cell sample, and a derivative or fraction thereof. In some embodiments, assaying the biological sample comprises sequencing the biological sample to generate the dataset. In some embodiments, the one or more computer processors are individually or collectively programmed to further identify the presence of the non-inflammatory disease or condition in the subject, or the likelihood that the subject will develop the non-inflammatory disease or condition, at a sensitivity of at least about 70%. In some embodiments, the one or more computer processors are individually or collectively programmed to further identify the presence of the non-inflammatory disease or condition in the subject, or the likelihood that the subject will develop the non-inflammatory disease or condition, at a sensitivity of at least about 80%. In some embodiments, the one or more computer processors are individually or collectively programmed to further identify the presence of the non-inflammatory disease or condition in the subject, or the likelihood that the subject will develop the non-inflammatory disease or condition, at a sensitivity of at least about 90%. In some embodiments, the one or more computer processors are individually or collectively programmed to further identify the presence of the non-inflammatory disease or condition in the subject, or the likelihood that the subject will develop the non-inflammatory disease or condition, at a sensitivity of at least about 95%. In some embodiments, the one or more computer processors are individually or collectively programmed to further identify the presence of the non-inflammatory disease or condition in the subject, or the likelihood that the subject will develop the non-inflammatory disease or condition, at a sensitivity of at least about 99%. In some embodiments, the one or more computer processors are individually or collectively programmed to further identify the presence of the non-inflammatory disease or condition in the subject, or the likelihood that the subject will develop the non-inflammatory disease or condition, at a specificity of at least about 70%. In some embodiments, the one or more computer processors are individually or collectively programmed to further identify the presence of the non-inflammatory disease or condition in the subject, or the likelihood that the subject will develop the non-inflammatory disease or condition, at a specificity of at least about 80%. In some embodiments, the one or more computer processors are individually or collectively programmed to further identify the presence of the non-inflammatory disease or condition in the subject, or the likelihood that the subject will develop the non-inflammatory disease or condition, at a specificity of at least about 90%. In some embodiments, the one or more computer processors are individually or collectively programmed to further identify the presence of the non-inflammatory disease or condition in the subject, or the likelihood that the subject will develop the non-inflammatory disease or condition, at a specificity of at least about 95%. In some embodiments, the one or more computer processors are individually or collectively programmed to further identify the presence of the non-inflammatory disease or condition in the subject, or the likelihood that the subject will develop the non-inflammatory disease or condition, at a specificity of at least about 99%. In some embodiments, the one or more computer processors are individually or collectively programmed to further identify the presence of the non-inflammatory disease or condition in the subject, or the likelihood that the subject will develop the non-inflammatory disease or condition, at a PPV of at least about 70%. In some embodiments, the one or more computer processors are individually or collectively programmed to further identify the presence of the non-inflammatory disease or condition in the subject, or the likelihood that the subject will develop the non-inflammatory disease or condition, at a PPV of at least about 80%. In some embodiments, the one or more computer processors are individually or collectively programmed to further identify the presence of the non-inflammatory disease or condition in the subject, or the likelihood that the subject will develop the non-inflammatory disease or condition, at a PPV of at least about 90%. In some embodiments, the one or more computer processors are individually or collectively programmed to further identify the presence of the non-inflammatory disease or condition in the subject, or the likelihood that the subject will develop the non-inflammatory disease or condition, at a PPV of at least about 95%. In some embodiments, the one or more computer processors are individually or collectively programmed to further identify the presence of the non-inflammatory disease or condition in the subject, or the likelihood that the subject will develop the non-inflammatory disease or condition, at a PPV of at least about 99%. In some embodiments, the one or more computer processors are individually or collectively programmed to further identify the presence of the non-inflammatory disease or condition in the subject, or the likelihood that the subject will develop the non-inflammatory disease or condition, at a negative predictive value (NPV) of at least about 70%. In some embodiments, the one or more computer processors are individually or collectively programmed to further identify the presence of the non-inflammatory disease or condition in the subject, or the likelihood that the subject will develop the non-inflammatory disease or condition, at a NPV of at least about 80%. In some embodiments, the one or more computer processors are individually or collectively programmed to further identify the presence of the non-inflammatory disease or condition in the subject, or the likelihood that the subject will develop the non-inflammatory disease or condition, at a NPV of at least about 90%. In some embodiments, the one or more computer processors are individually or collectively programmed to further identify the presence of the non-inflammatory disease or condition in the subject, or the likelihood that the subject will develop the non-inflammatory disease or condition, at a NPV of at least about 95%. In some embodiments, the one or more computer processors are individually or collectively programmed to further identify the presence of the non-inflammatory disease or condition in the subject, or the likelihood that the subject will develop the non-inflammatory disease or condition, at a NPV of at least about 99%. In some embodiments, the one or more computer processors are individually or collectively programmed to further identify the presence of the non-inflammatory disease or condition in the subject, or the likelihood that the subject will develop the non-inflammatory disease or condition, with an Area Under Curve (AUC) of at least about 0.70. In some embodiments, the one or more computer processors are individually or collectively programmed to further identify the presence of the non-inflammatory disease or condition in the subject, or the likelihood that the subject will develop the non-inflammatory disease or condition, with an AUC of at least about 0.80. In some embodiments, the one or more computer processors are individually or collectively programmed to further identify the presence of the non-inflammatory disease or condition in the subject, or the likelihood that the subject will develop the non-inflammatory disease or condition, with an AUC of at least about 0.90. In some embodiments, the one or more computer processors are individually or collectively programmed to further identify the presence of the non-inflammatory disease or condition in the subject, or the likelihood that the subject will develop the non-inflammatory disease or condition, with an AUC of at least about 0.95. In some embodiments, the one or more computer processors are individually or collectively programmed to further identify the presence of the non-inflammatory disease or condition in the subject, or the likelihood that the subject will develop the non-inflammatory disease or condition, with an AUC of at least about 0.99. In some embodiments, the subject is asymptomatic for one or more non-inflammatory disease or conditions. In some embodiments, the deep learning prediction model is trained using a first set of independent training samples associated with a presence of the non-inflammatory disease or condition and a second set of independent training samples associated with an absence of the non-inflammatory disease or condition. In some embodiments, the one or more computer processors are individually or collectively further programmed to apply the deep learning prediction model (e.g., a deep learning classifier) to a set of clinical health data of the subject. In some embodiments, the set of clinical health data comprises one or more of familial history of an non-inflammatory disease or disorder, age, hypertension or pre-hypertension, diabetes or pre-diabetes, overweight or obesity, environmental exposure, lifestyle risk factors (e.g., smoking, alcohol consumption, or drug use), or presence of other risk factors. In some embodiments, the deep learning prediction model comprises a deep learning algorithm, a neural network, a Random Forest, an XGBoost, or a Gradient Boost. In some embodiments, the deep learning prediction model comprises a deep learning algorithm. In some embodiments, the deep learning algorithm comprises a deep neural network. In some embodiments, the deep neural network comprises a convolutional neural network (CNN). In some embodiments, the one or more computer processors are individually or collectively programmed to further optimize a set of hyperparameters of the CNN. In some embodiments, optimizing the set of hyperparameters comprises performing an intensive grid search. In some embodiments, the set of hyperparameters comprises a number of layers and/or a number of neurons of the CNN. In some embodiments, the CNN comprises a combination of a plurality of CNNs. In some embodiments, the plurality of CNNs comprises two CNNs. In some embodiments, assaying the biological sample comprises subjecting the biological sample to conditions that are sufficient to isolate, enrich, or extract a plurality of DNA molecules; and analyzing the plurality of DNA molecules to generate the dataset. In some embodiments, the plurality of genomic loci comprises at least about 1,000 distinct genomic loci. In some embodiments, the plurality of genomic loci comprises at least about 10,000 distinct genomic loci. In some embodiments, the plurality of genomic loci comprises at least about 100,000 distinct genomic loci. In some embodiments, the one or more computer processors are individually or collectively programmed to further identify the likelihood that the subject will develop the non-inflammatory disease or condition. In some embodiments, the one or more computer processors are individually or collectively programmed to further provide a therapeutic intervention for the non-inflammatory disease or condition, provided the presence of the non-inflammatory disease or condition is identified in the subject. In some embodiments, the one or more computer processors are individually or collectively programmed to further monitor the non-inflammatory disease or condition in the subject by assessing the non-inflammatory disease or condition of the subject at a plurality of time points, wherein the assessing is based at least partially on identifying the presence of the non-inflammatory disease or condition in (ii) by the one or more computer processors at one or more time points of the plurality of time points. In some embodiments, a difference between two or more assessments of the non-inflammatory disease or condition in the subject at two or more time points of the plurality of time points is indicative of one or more of: (i) a diagnosis of the non-inflammatory disease or condition of the subject, (ii) a prognosis of the non-inflammatory disease or condition of the subject, and (iii) an efficacy or non-efficacy of a course of treatment for treating the non-inflammatory disease or condition of the subject. In some embodiments, the system further comprises an electronic display operatively coupled to the one or more computer processors, wherein the electronic display comprises a graphical user interface that is configured to display the report.

In another aspect, the present disclosure provides a computer system for identifying a fibrotic disease in a subject, comprising: (a) a database that is configured to store a dataset comprising genetic data, wherein the genetic data is obtained by assaying a biological sample of the subject; and (b) one or more computer processors operatively coupled to the database, wherein the one or more computer processors are individually or collectively programmed to: (i) process the dataset at a plurality of genomic loci to determine quantitative measures of each genomic locus of the plurality of genomic loci, wherein the plurality of genomic loci comprises fibrotic disease-associated genes, thereby producing a fibrotic disease profile of the biological sample of the subject; and (ii) apply a deep learning prediction model to the fibrotic disease profile to identify a presence of the fibrotic disease in the subject, or a likelihood that the subject will develop the fibrotic disease. In some embodiments, the fibrotic disease comprises Primary Sclerosing Cholangitis (PSC), scleroderma, or pulmonary fibrosis. In some embodiments, the fibrotic disease comprises the PSC. In some embodiments, the fibrotic disease comprises the scleroderma. In some embodiments, the fibrotic disease comprises the pulmonary fibrosis. In some embodiments, the diabetes comprises type 2 diabetes. In some embodiments, the neurological disease comprises Alzheimer's disease. In some embodiments, the biological sample is selected from the group consisting of: a whole blood sample, a DNA sample, a RNA sample, a cell-free sample, a tissue sample, a cell sample, and a derivative or fraction thereof. In some embodiments, assaying the biological sample comprises sequencing the biological sample to generate the dataset. In some embodiments, the one or more computer processors are individually or collectively programmed to further identify the presence of the fibrotic disease in the subject, or the likelihood that the subject will develop the fibrotic disease, at a sensitivity of at least about 70%. In some embodiments, the one or more computer processors are individually or collectively programmed to further identify the presence of the fibrotic disease in the subject, or the likelihood that the subject will develop the fibrotic disease, at a sensitivity of at least about 80%. In some embodiments, the one or more computer processors are individually or collectively programmed to further identify the presence of the fibrotic disease in the subject, or the likelihood that the subject will develop the fibrotic disease, at a sensitivity of at least about 90%. In some embodiments, the one or more computer processors are individually or collectively programmed to further identify the presence of the fibrotic disease in the subject, or the likelihood that the subject will develop the fibrotic disease, at a sensitivity of at least about 95%. In some embodiments, the one or more computer processors are individually or collectively programmed to further identify the presence of the fibrotic disease in the subject, or the likelihood that the subject will develop the fibrotic disease, at a sensitivity of at least about 99%. In some embodiments, the one or more computer processors are individually or collectively programmed to further identify the presence of the fibrotic disease in the subject, or the likelihood that the subject will develop the fibrotic disease, at a specificity of at least about 70%. In some embodiments, the one or more computer processors are individually or collectively programmed to further identify the presence of the fibrotic disease in the subject, or the likelihood that the subject will develop the fibrotic disease, at a specificity of at least about 80%. In some embodiments, the one or more computer processors are individually or collectively programmed to further identify the presence of the fibrotic disease in the subject, or the likelihood that the subject will develop the fibrotic disease, at a specificity of at least about 90%. In some embodiments, the one or more computer processors are individually or collectively programmed to further identify the presence of the fibrotic disease in the subject, or the likelihood that the subject will develop the fibrotic disease, at a specificity of at least about 95%. In some embodiments, the one or more computer processors are individually or collectively programmed to further identify the presence of the fibrotic disease in the subject, or the likelihood that the subject will develop the fibrotic disease, at a specificity of at least about 99%. In some embodiments, the one or more computer processors are individually or collectively programmed to further identify the presence of the fibrotic disease in the subject, or the likelihood that the subject will develop the fibrotic disease, at a PPV of at least about 70%. In some embodiments, the one or more computer processors are individually or collectively programmed to further identify the presence of the fibrotic disease in the subject, or the likelihood that the subject will develop the fibrotic disease, at a PPV of at least about 80%. In some embodiments, the one or more computer processors are individually or collectively programmed to further identify the presence of the fibrotic disease in the subject, or the likelihood that the subject will develop the fibrotic disease, at a PPV of at least about 90%. In some embodiments, the one or more computer processors are individually or collectively programmed to further identify the presence of the fibrotic disease in the subject, or the likelihood that the subject will develop the fibrotic disease, at a PPV of at least about 95%. In some embodiments, the one or more computer processors are individually or collectively programmed to further identify the presence of the fibrotic disease in the subject, or the likelihood that the subject will develop the fibrotic disease, at a PPV of at least about 99%. In some embodiments, the one or more computer processors are individually or collectively programmed to further identify the presence of the fibrotic disease in the subject, or the likelihood that the subject will develop the fibrotic disease, at a negative predictive value (NPV) of at least about 70%. In some embodiments, the one or more computer processors are individually or collectively programmed to further identify the presence of the fibrotic disease in the subject, or the likelihood that the subject will develop the fibrotic disease, at a NPV of at least about 80%. In some embodiments, the one or more computer processors are individually or collectively programmed to further identify the presence of the fibrotic disease in the subject, or the likelihood that the subject will develop the fibrotic disease, at a NPV of at least about 90%. In some embodiments, the one or more computer processors are individually or collectively programmed to further identify the presence of the fibrotic disease in the subject, or the likelihood that the subject will develop the fibrotic disease, at a NPV of at least about 95%. In some embodiments, the one or more computer processors are individually or collectively programmed to further identify the presence of the fibrotic disease in the subject, or the likelihood that the subject will develop the fibrotic disease, at a NPV of at least about 99%. In some embodiments, the one or more computer processors are individually or collectively programmed to further identify the presence of the fibrotic disease in the subject, or the likelihood that the subject will develop the fibrotic disease, with an Area Under Curve (AUC) of at least about 0.70. In some embodiments, the one or more computer processors are individually or collectively programmed to further identify the presence of the fibrotic disease in the subject, or the likelihood that the subject will develop the fibrotic disease, with an AUC of at least about 0.80. In some embodiments, the one or more computer processors are individually or collectively programmed to further identify the presence of the fibrotic disease in the subject, or the likelihood that the subject will develop the fibrotic disease, with an AUC of at least about 0.90. In some embodiments, the one or more computer processors are individually or collectively programmed to further identify the presence of the fibrotic disease in the subject, or the likelihood that the subject will develop the fibrotic disease, with an AUC of at least about 0.95. In some embodiments, the one or more computer processors are individually or collectively programmed to further identify the presence of the fibrotic disease in the subject, or the likelihood that the subject will develop the fibrotic disease, with an AUC of at least about 0.99. In some embodiments, the subject is asymptomatic for one or more fibrotic diseases. In some embodiments, the deep learning prediction model is trained using a first set of independent training samples associated with a presence of the fibrotic disease and a second set of independent training samples associated with an absence of the fibrotic disease. In some embodiments, the one or more computer processors are individually or collectively further programmed to apply the deep learning prediction model (e.g., a deep learning classifier) to a set of clinical health data of the subject. In some embodiments, the set of clinical health data comprises one or more of familial history of an non-inflammatory disease or disorder, age, hypertension or pre-hypertension, diabetes or pre-diabetes, overweight or obesity, environmental exposure, lifestyle risk factors (e.g., smoking, alcohol consumption, or drug use), or presence of other risk factors. In some embodiments, the deep learning prediction model comprises a deep learning algorithm, a neural network, a Random Forest, an XGBoost, or a Gradient Boost. In some embodiments, the deep learning prediction model comprises a deep learning algorithm. In some embodiments, the deep learning algorithm comprises a deep neural network. In some embodiments, the deep neural network comprises a convolutional neural network (CNN). In some embodiments, the one or more computer processors are individually or collectively programmed to further optimize a set of hyperparameters of the CNN. In some embodiments, optimizing the set of hyperparameters comprises performing an intensive grid search. In some embodiments, the set of hyperparameters comprises a number of layers and/or a number of neurons of the CNN. In some embodiments, the CNN comprises a combination of a plurality of CNNs. In some embodiments, the plurality of CNNs comprises two CNNs. In some embodiments, assaying the biological sample comprises subjecting the biological sample to conditions that are sufficient to isolate, enrich, or extract a plurality of DNA molecules; and analyzing the plurality of DNA molecules to generate the dataset. In some embodiments, the plurality of genomic loci comprises at least about 1,000 distinct genomic loci. In some embodiments, the plurality of genomic loci comprises at least about 10,000 distinct genomic loci. In some embodiments, the plurality of genomic loci comprises at least about 100,000 distinct genomic loci. In some embodiments, the one or more computer processors are individually or collectively programmed to further identify the likelihood that the subject will develop the fibrotic disease. In some embodiments, the one or more computer processors are individually or collectively programmed to further provide a therapeutic intervention for the fibrotic disease, provided the presence of the fibrotic disease is identified in the subject. In some embodiments, the one or more computer processors are individually or collectively programmed to further monitor the fibrotic disease in the subject by assessing the fibrotic disease of the subject at a plurality of time points, wherein the assessing is based at least partially on identifying the presence of the fibrotic disease in (ii) by the one or more computer processors at one or more time points of the plurality of time points. In some embodiments, a difference between two or more assessments of the fibrotic disease in the subject at two or more time points of the plurality of time points is indicative of one or more of: (i) a diagnosis of the fibrotic disease of the subject, (ii) a prognosis of the fibrotic disease of the subject, and (iii) an efficacy or non-efficacy of a course of treatment for treating the fibrotic disease of the subject.

Aspects disclosed herein provide non-transitory computer-readable media comprising machine-executable code that, upon execution by one or more computer processors, implements a method for identifying an non-inflammatory disease or condition of a subject, the method comprising: (a) assaying a biological sample of the subject to generate a dataset comprising genetic data; (b) processing the dataset at a plurality of genomic loci to determine quantitative measures of each of the plurality of genomic loci, wherein the plurality of genomic loci comprises non-inflammatory disease-associated genes, thereby producing an non-inflammatory disease profile of the biological sample of the subject; and (c) applying a deep learning prediction model to the non-inflammatory disease profile to identify a presence or an absence of the non-inflammatory disease or condition in the subject, or a risk that the subject will develop the non-inflammatory disease or condition.

In another aspect, the present disclosure provides a non-transitory computer-readable media comprising machine-executable code that, upon execution by one or more computer processors, implements a method for identifying a fibrotic disease of a subject, the method comprising: (a) assaying a biological sample of the subject to generate a dataset comprising genetic data; (b) processing the dataset at a plurality of genomic loci to determine quantitative measures of each of the plurality of genomic loci, wherein the plurality of genomic loci comprises fibrotic disease-associated genes, thereby producing an fibrotic disease profile of the biological sample of the subject; and (c) applying a deep learning prediction model to the fibrotic disease profile to identify a presence of the fibrotic disease in the subject, or a risk that the subject will develop the fibrotic disease.

Aspects disclosed herein provide non-transitory computer-readable media comprising machine-executable code that, upon execution by one or more computer processors, implements any of the methods above or elsewhere herein.

Aspects disclosed herein provide systems comprising one or more computer processors and computer memory coupled thereto. The computer memory comprises machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a non-limiting example of a workflow to profile non-inflammatory diseases or conditions (e.g., fibrotic disease) via deep learning approaches, using the methods and systems disclosed herein.

FIG. 2 shows a non-limiting example of a computer system that is programmed to implement methods of the disclosure.

FIG. 3 shows a non-limiting example of a DeepLearning algorithm based on neural networking (similar to a brain's neurons), using the methods and systems disclosed herein.

FIG. 4 shows a non-limiting example of DeepLearning algorithms using deep layers of neurons having an input layer, an output layer, and multiple intermediate layers between the input and output layers, using the methods and systems disclosed herein.

FIG. 5 shows a non-limiting example of activation functions (e.g., fixed mathematical operations) that may be used in DeepLearning algorithms, such as sigmoid, tanh, ReLU, leaky ReLU, maxout, and ELU, using the methods and systems disclosed herein.

FIGS. 6A-6B show non-limiting examples of forward propagation (FIG. 6A) and backpropagation (FIG. 6B) of a DeepLearning algorithm, using the methods and systems disclosed herein.

### DETAILED DESCRIPTION

Non-inflammatory diseases and disorders may have a significant effect on morbidity and quality of life. Non-inflammatory diseases and disorders affect millions of people in the United States. The significant effect on morbidity is, in part, due to limitations of existing diagnostic and prognostic tests that fail to identify patients suffering from non-inflammatory diseases early enough in disease progression to prevent worsening of the disease or development of complications, such as certain severe or advanced-stage disease phenotypes.

Delay in disease diagnosis or prognosis is a major clinical problem. Early therapeutic intervention of non-inflammatory diseases in patients at high risk for developing severe forms of the disease may lead to lower risk of tissue damage in the affected area, significantly improved disease remission, fewer disease complications, and a reduced need for surgery. For many patients suffering from non-inflammatory disease, early therapeutic intervention is associated with a higher response to prescribed medication to treat the disease. Early therapeutic interventions include, but are not limited to, active agents that modulate the gut microbiome or targeted (e.g., biologic therapies).

There have been recent efforts to predict complex disease risk using genetic data with the LDpred approach, a Python based software package that adjusts genome-wide association study (GWAS) summary statistics for the effects of linkage disequilibrium (LD) and, in some cases, incorporates variants that have not reached a genome-wide significance threshold. However, the LDpred approach may suffer at least from the following drawbacks. The LDpred approach may not perform stringent quality control procedures to prune the input datasets, which may adversely affect the performance. The LDpred approach may not make use of convolutional neural networks, which automatically include two data pre-processing layers (Convolutional Layer and Pooling Layer) that perform much of the computational heavy lifting before the fully-connected layers. The LDpred approach may comprise a manual single nucleotide polymorphism (SNP) preselection step based on single-SNP level statistics may be performed to reduce the dimension of data, which may potentially lead to loss of information. The LDpred approach may not comprise intensive tuning of a set of hyperparameters which may have important impact on performance of the models. The LDpred approach may not use a superlearner that is constructed by combining the two separately trained models. The LDpred approach may fail to account for non-linear effects among known variants.

Provided herein are systems and methods that apply deep learning approaches to predict complex disease risk. The deep learning approaches described herein analyze genetic data of a subject to identify the subject as having a presence or an absence of, or being at high risk of having, or developing, an non-inflammatory disease (e.g., fibrotic disease). The deep learning approaches described herein utilize prediction tools from a broader family of machine learning methods with proven records in prediction performance. The present disclosure provides a comparison between the performance of the deep learning and LDpred approaches to show the superior clinical utility (e.g., for clinical decision-making or assessment) of the deep learning approach described herein.

Provided herein are methods and systems for predicting that a subject has, or will develop, an non-inflammatory disease (e.g., fibrotic disease) using a DeepLearning (DL) model. The DL model is useful for the diagnosis, prognosis, monitoring, treatment, or prevention of an non-inflammatory disease described herein. The DL model is useful for identifying a subject at a high risk for developing a severe form of the non-inflammatory disease described herein, including complications (e.g., severe, advanced-stage, or medically refractory disease phenotypes). The DL model is useful for monitoring a course of treatment of a subject to optimize or tailor a therapeutic intervention to a particular subject.

In contrast to LDpred approach, the DL model described herein perform stringent quality control procedures to prune the input datasets. The DL model described herein also utilize convolutional neural networks that do not require a preselection of SNPs and are capable of accounting for non-linear effects of genetic variants. All of the above, in combination with intensive tuning of the hyperparameters of the deep learning algorithms utilized in the DL model described herein, ensure a more accurate and more efficient prediction, as compared to the predictions generated using LDpred.

Using methods and systems of the present disclosure, the DL model employs deep learning algorithms to analyze genetic data of a subject. Such deep learning algorithms significantly boost prediction accuracy and associate the predicted risk for disease with disease clinical characteristics. The clinical utility of the methods and systems of the present disclosure is underscored by the ability of the DL model to analyze large-scale genomic data, such as next-generation sequencing (NGS) data, to predict a wide range of non-inflammatory diseases. The DL model described herein applied to large-scale genomic data may translate into clinical practice, by aiding medical practitioners in providing individualized therapeutic strategies for the treatment of complex disease, such as the non-inflammatory diseases described herein.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms may include quantitative, qualitative or quantitative and qualitative determinations. Assessing may be relative or absolute. “Detecting the presence of” may include determining the amount of something present in addition to determining whether it is present or absent depending on the context.

The terms “subject,” or “individual,” are often used interchangeably herein. A “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at elevated or high risk for an non-inflammatory disease. A subject diagnosed with an non-inflammatory disease or condition disclosed herein may be referred to as a “patient.” In some cases, the subject is not necessarily diagnosed or suspected of being at high risk for the non-inflammatory disease. The subject may be displaying a symptom(s) indicative of a health or physiological state or condition of the subject, such as an non-inflammatory disease or disorder of the subject. As an alternative, the subject may be asymptomatic with respect to such health or physiological state or condition. For example, the subject may be asymptomatic with respect to an non-inflammatory disease or condition, characterized by an absence of symptoms associated with the non-inflammatory disease or condition (e.g., pain, fatigue, nausea, weight loss, weakness, bleeding, and loss of function).

A “genetic variant” as used herein refers to an aberration in a nucleic acid sequence, as compared to the nucleic acid sequence in a reference population. In some cases, the aberration is a polymorphism, such as a single nucleotide polymorphism or an indel.

As used herein, the term, “single nucleotide polymorphism” or “SNP,” refers to a variation in a single nucleotide within a polynucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic.

The term, “indel,” as disclosed herein, refers to an insertion, or a deletion, of a nucleobase within a polynucleotide sequence.

As used herein, the term “non-inflammatory disease” refers to a disease, disorder, or other abnormal condition of a subject that belongs to a class of diseases or disorders that are not proven to be predominantly caused by chronic inflammation. The non-inflammatory disease may be characterized by a combination of one or more symptoms in the subject, including pain, fatigue, nausea, weight loss, weakness, bleeding, and loss of function. The non-inflammatory disease may be characterized as a severe or advanced-stage form of the disease. The non-inflammatory disease may be characterized as a mild or early-stage form of the disease. The non-inflammatory disease may be medically refractory. The non-inflammatory disease may be a fibrotic disease or condition.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r2 value corresponding to the difference between an observed and expected unit of risk frequencies in the population (D=Pab−PaPb), which is scaled by the individual frequencies of the different loci.

As used herein, the term “medically refractory” refers to a disease, disorder, or other abnormal condition of a subject that is non-responsive to a standard therapy, such as a drug Non-limiting examples of standard therapy include drugs or other treatments suitable for a non-inflammatory disease or disorder, such as cardiovascular disease, adolescent idiopathic scoliosis, a neurological disease, a fibrotic disease (e.g., PSC, scleroderma, or pulmonary fibrosis), type 2 diabetes, Alzheimer's disease, and obesity.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of an non-inflammatory disease or condition, delaying or eliminating the onset of symptoms of an non-inflammatory disease or condition, slowing, halting, or reversing the progression of an non-inflammatory disease or condition, or any combination thereof. For prophylactic benefit, a subject at risk of developing a particular non-inflammatory disease, or to a subject reporting one or more of the physiological symptoms of an non-inflammatory disease may undergo treatment, even though a diagnosis of this non-inflammatory disease may not have been made.

As used herein the term “diagnose” or “diagnosis” of a status or outcome includes predicting or diagnosing the status or outcome, determining predisposition to a status or outcome, monitoring treatment of patient, diagnosing a therapeutic response of a patient, and prognosis of status or outcome, progression, and response to particular treatment.

As used herein, the term “biological sample,” generally refers to a biological sample obtained from or derived from one or more subjects from which nucleic acids may be obtained. Non-limiting examples of a “biological sample” include whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. The biological sample can be obtained through primary patient derived cell lines, or archived patient samples in the form of preserved samples, or fresh frozen samples. The biological sample may be a deoxyribonucleic acid (DNA) sample or a ribonucleic acid (RNA) sample, which refers to any biological sample above containing DNA and/or RNA that has been at least partially purified and/or isolated.

The term “derived from” used herein refers to an origin or source, and may include naturally occurring, recombinant, unpurified, or purified molecules.

To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with an non-inflammatory disease or disorder. Samples may be obtained from a subject during a treatment or a treatment regime. Multiple samples may be obtained from a subject to monitor the effects of the treatment over time. The sample may be taken from a subject known or suspected of having an non-inflammatory disease or disorder for which a definitive positive or negative diagnosis is not available via clinical tests. The sample may be taken from a subject suspected of having an non-inflammatory disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing an non-inflammatory disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of an non-inflammatory disease. In some embodiments, the progression of an non-inflammatory disease may be tracked before treatment, after treatment, or during the course of treatment, to determine the treatment's effectiveness. For example, a method as described herein may be performed on a subject prior to, and after, treatment of a subject with an non-inflammatory disease therapy to measure the subject's disease progression or regression in response to the non-inflammatory disease therapy.

After obtaining a sample from the subject, the sample may be processed to generate datasets indicative of an non-inflammatory disease or disorder of the subject. For example, a presence, absence, or quantitative assessment of nucleic acid molecules of the sample at a panel of non-inflammatory disease-associated genomic loci may be indicative of an non-inflammatory disease of the subject. For example, the non-inflammatory disease-associated genomic loci may have been shown to be correlated with presence or risk of an non-inflammatory disease (e.g., as shown through GWAS statistics). The nucleic acid molecules may comprise ribonucleic acid (RNA) or deoxyribonucleic acid (DNA).

Processing the sample obtained from the subject may comprise (i) subjecting the sample to conditions that are sufficient to isolate, enrich, or extract a plurality of nucleic acid molecules (e.g., DNA or RNA), and (ii) assaying the plurality of nucleic acid molecules (e.g., DNA or RNA) to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

The term “cell-free nucleic acid (cfNA)”, as used herein, generally refers to nucleic acids (such as cell-free RNA (“cfRNA”) or cell-free DNA (“cfDNA”)) in a biological sample that are not contained in a cell. cfDNA may circulate freely in in a bodily fluid, such as in the bloodstream.

The term “cell-free sample”, as used herein, generally refers to a biological sample that is substantially devoid of intact cells. This may be derived from a biological sample that is itself substantially devoid of cells or may be derived from a sample from which cells have been removed. Examples of cell-free samples include those derived from blood, such as serum or plasma; urine; or samples derived from other sources, such as semen, sputum, feces, ductal exudate, lymph, or recovered lavage.

The term “genomic region” or “genomic locus”, as used interchangeably herein, generally refers to identified regions of nucleic acid that are identified by their location in the chromosome. In some examples, the genomic regions are referred to by a gene name and encompass coding and non-coding regions associated with that physical region of nucleic acid. As used herein, a gene comprises coding regions (exons), non-coding regions (introns), transcriptional control or other regulatory regions, and promoters. In another example, the genomic region may incorporate an intron or exon or an intron/exon boundary within a named gene.

The term “confidence interval” or “CI”, as used interchangeably herein, generally refers to a range of values which contains an unknown parameter (e.g., mean) of a set of observations with a given level of confidence or certainty. For example, a 95% CI may refer to a range of values which contains the true mean of a set of observations with a 95% confidence.

The section headings used herein are for organizational purposes only and are not to be construed as limiting the subject matter described.

### II. METHODS

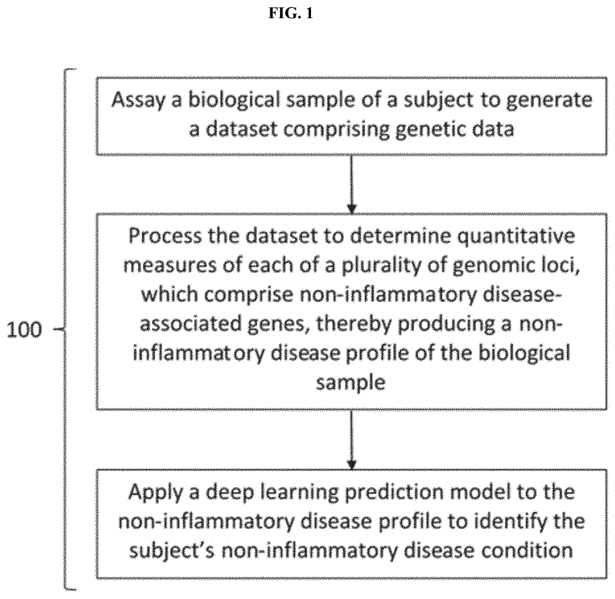

FIG. 1 shows a non-limiting example of a workflow to profile non-inflammatory diseases or conditions via deep learning approaches, using the methods and systems disclosed herein. In an aspect, the present disclosure provides a method 100 for identifying an non-inflammatory disease or condition of a subject, comprising: assaying a biological sample of the subject to generate a dataset comprising genetic data (as in step 102); processing the dataset at a plurality of genomic loci to determine quantitative measures of each of the genomic loci, wherein the plurality of genomic loci comprises non-inflammatory disease-associated genes, thereby producing an non-inflammatory disease profile of the biological sample of the subject (as in step 104); and applying a deep learning prediction model to the non-inflammatory disease profile to identify the non-inflammatory disease or condition of the subject (as in step 106). For example, the non-inflammatory disease profile may comprise a plurality of quantitative measures of each of a plurality of non-inflammatory disease-associated genomic loci and/or a set of clinical health data of the subject. In some embodiments, the set of clinical health data comprises one or more of familial history of an non-inflammatory disease or disorder, age, hypertension or pre-hypertension, diabetes or pre-diabetes, overweight or obesity, environmental exposure, lifestyle risk factors (e.g., smoking, alcohol consumption, or drug use), or presence of other risk factors.

The biological samples may be obtained or derived from a human subject (e.g., a subject having or suspected of having an non-inflammatory disease or disorder). The biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, RNA collection tubes, or DNA collection tubes).

The biological sample may be obtained from a subject with an non-inflammatory disease, disorder, or condition, from a subject that is suspected of having an non-inflammatory disease, disorder, or condition, or from a subject that does not have or is not suspected of having the non-inflammatory disease, disorder, or condition.

The non-inflammatory disease may include, but is not limited to, one or more of: cardiovascular disease, adolescent idiopathic scoliosis, a neurological disease, a fibrotic disease (e.g., PSC, scleroderma, or pulmonary fibrosis), type 2 diabetes, Alzheimer's disease, and obesity.

The non-inflammatory disease may be treated with a variety of treatments, such as drugs, analgesics (e.g., acetaminophen), herbal supplements, and other suitable supplements.

In some embodiments, the non-inflammatory disease or condition may comprise a likelihood, risk, or susceptibility of having an non-inflammatory disease in the future (e.g., within about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, or more than about 10 years).

The biological sample may be taken before and/or after treatment of a subject with the non-inflammatory disease or condition. Biological samples may be obtained from a subject during a treatment or a treatment regime. Multiple biological samples may be obtained from a subject to monitor the effects of the treatment over time. The biological sample may be taken from a subject known or suspected of having an non-inflammatory disease or condition for which a definitive positive or negative diagnosis is not available via clinical tests. The sample may be taken from a subject suspected of having an non-inflammatory disease or condition. The biological sample may be taken from a subject experiencing unexplained symptoms, such as fatigue, nausea, weight loss, aches and pains, weakness, or bleeding. The biological sample may be taken from a subject having explained symptoms. The biological sample may be taken from a subject at risk of developing an non-inflammatory disease or condition due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The biological sample may contain one or more analytes capable of being assayed, such as deoxyribonucleic acid (DNA) molecules suitable for assaying to generate genomic data, ribonucleic acid (RNA) molecules suitable for assaying to generate transcriptomic data, proteins suitable for assaying to generate proteomic data, metabolites suitable for assaying to generate metabolomic data, or a mixture or combination thereof. One or more such analytes (e.g., DNA molecules, RNA molecules, proteins, and/or metabolites) may be isolated or extracted from one or more biological samples of a subject for downstream assaying using one or more suitable assays.

After obtaining a biological sample from the subject, the biological sample may be processed to generate datasets indicative of an non-inflammatory disease or condition of the subject. For example, a presence, absence, or quantitative assessment of nucleic acid molecules of the biological sample at a panel of non-inflammatory disease-associated genomic loci (e.g., quantitative measures of DNA or RNA at the non-inflammatory disease-associated genomic loci), proteomic data comprising quantitative measures of proteins of the dataset at a panel of non-inflammatory disease-associated proteins, and/or metabolome data comprising quantitative measures of a panel of non-inflammatory disease-associated metabolites may be indicative of an non-inflammatory disease-associated. Processing the biological sample obtained from the subject may comprise (i) subjecting the biological sample to conditions that are sufficient to isolate, enrich, or extract a plurality of nucleic acid molecules, proteins, and/or metabolites, and (ii) assaying the plurality of nucleic acid molecules, proteins, and/or metabolites to generate the dataset.

In some embodiments, a plurality of nucleic acid molecules is extracted from the biological sample and subjected to sequencing to generate a plurality of sequencing reads. The nucleic acid molecules may comprise deoxyribonucleic acid (DNA) or ribonucleic acid (RNA). The nucleic acid molecules (e.g., DNA or RNA) may be extracted from the biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all DNA or RNA molecules from a sample. Alternatively, the extract method may selectively extract a portion of DNA or RNA molecules from a sample. Extracted RNA molecules from a sample may be converted to cDNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing by binding, sequencing-by-ligation, sequencing-by-hybridization, and RNA-Seq (Illumina). The sequencing may comprise unbiased sequencing, such as whole genome sequencing (WGS). The sequencing may comprise targeted sequencing, with higher sequencing depth or targeted enrichment of a plurality of non-inflammatory disease-associated genomic loci.

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with pregnancy-related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

DNA or RNA molecules isolated or extracted from a biological sample may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial biological samples. For example, a plurality of biological samples may be tagged with sample barcodes such that each DNA molecule may be traced back to the sample (and the subject) from which the DNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the data indicative of the presence, absence, or relative assessment of the non-inflammatory disease-associated genomic loci. For example, the sequence reads may be aligned to one or more reference genomes (e.g., a genome of one or more species such as a human genome). The aligned sequence reads may be quantified at one or more genomic loci to generate the datasets indicative of the non-inflammatory disease. For example, quantification of sequences corresponding to a plurality of genomic loci associated with non-inflammatory disease may generate the datasets indicative of the non-inflammatory disease.

The biological sample may be processed without any nucleic acid extraction. For example, the non-inflammatory disease may be identified or monitored in the subject by using probes configured to selectively enrich nucleic acid (e.g., DNA or RNA) molecules corresponding to the plurality of non-inflammatory disease-associated genomic loci. The probes may be nucleic acid primers. The probes may have sequence complementarity with nucleic acid sequences from one or more of the plurality of non-inflammatory disease-associated genomic loci or genomic regions. The plurality of non-inflammatory disease-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct non-inflammatory disease-associated genomic loci or genomic regions.

The probes may be nucleic acid molecules (e.g., DNA or RNA) having sequence complementarity with nucleic acid sequences (e.g., DNA or RNA) of the one or more genomic loci (e.g., non-inflammatory disease-associated genomic loci). These nucleic acid molecules may be primers or enrichment sequences. The assaying of the biological sample using probes that are selective for the one or more genomic loci (e.g., non-inflammatory disease-associated genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., non-inflammatory disease-associated genomic loci) to generate the data indicative of the non-inflammatory disease. For example, quantification of array hybridization or polymerase chain reaction (PCR) corresponding to a plurality of genomic loci (e.g., non-inflammatory disease-associated genomic loci) may generate data indicative of the non-inflammatory disease. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

The biological samples may be processed using a methylation-specific assay. For example, a methylation-specific assay may be used to identify a quantitative measure (e.g., indicative of a presence, absence, or relative amount) of methylation each of a plurality of non-inflammatory disease-associated genomic loci in a biological sample of the subject. The methylation-specific assay may be configured to process biological samples such as a blood sample or a urine sample (or derivatives thereof) of the subject. A quantitative measure (e.g., indicative of a presence, absence, or relative amount) of methylation of non-inflammatory disease-associated genomic loci in the biological sample may be indicative of one or more non-inflammatory diseases. The methylation-specific assay may be used to generate datasets indicative of the quantitative measure (e.g., indicative of a presence, absence, or relative amount) of methylation of each of a plurality of non-inflammatory disease-associated genomic loci in the biological sample of the subject.

The methylation-specific assay may comprise, for example, one or more of: a methylation-aware sequencing (e.g., using bisulfite treatment), pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

Subject recruitment for a cohort having a given non-inflammatory disease may be performed as follows. A first number of patients with the given non-inflammatory disease and a second number of control subjects without the given non-inflammatory disease may be recruited from a variety of geographic locations. Diagnosis of the given non-inflammatory disease may be performed based on accepted radiological, histopathological, and other clinical evaluation. All included cases may fulfill clinical criteria for the given non-inflammatory disease. Written informed consent may be obtained from all study participants. The entire cohort may be used as a training set in the current investigation.

A first number of non-inflammatory disease cases with genotype data (after QC) may be included as cases in the test set cohort. The diagnosis of each patient may be performed based on standard histologic, radiographic, and other features. Blood samples may be collected at the time of enrollment. The study protocol and data collection, including DNA preparation and genotyping, may be approved by an Institutional Review Board. Written informed consent may be obtained from all study participants.

Genotyping and genotype quality control (QC) may be performed as follows. Genotyping of the test set cohort may be performed using an Illumina ImmunoChip array. Individual and genotype missingness, allele frequencies, and deviations from Hardy-Weinberg Equilibrium may be calculated using the PLINK software package (pngu.mgh.harvard.edu/˜purcell/plink). Individual-level QC thresholds may include a high genotyping call rate (e.g., greater than 95%) and a low inbreeding coefficient (e.g., less than 0.05). Ethnicity outliers may be identified using Admixture software and may be removed. Single nucleotide polymorphisms (SNPs) with a low call rate (e.g., less than 0.95), with a low minor allele frequency (MAF) (e.g., less than 0.01), and that strongly deviated from Hardy-Weinberg equilibrium (e.g., p<1×10⁻⁷) may also be removed.

Genotyping and QC in the non-inflammatory disease cohort may be performed as follows. In brief, the Immunochip samples may be genotyped in 36 batches, and genotype calling may be performed separately for each batch. Similar QC may be performed, which removes SNPs with a call rate lower than 98% across all genotyping batches or 90% in one of the genotyping batches, not in 1000 Genomes Project Phase I, failing Hardy-Weinberg Equilibrium (FDR<1×10⁻⁵ across all samples or within each genotyping batch), or monomorphic SNPs. Individuals may be assigned to different populations based on principal components and those not in the European Ancestry cluster, with a low call rate (e.g., less than 98%), outlying heterozygosity rate (e.g., FDR less than 0.01) or cryptic relatedness (e.g., identity by decent greater than 0.4) may be removed.

A set of SNPs that passed the QC in both the non-inflammatory disease cohort and the test set cohort may be included in current analysis. Of those SNPs, a first number may be known non-inflammatory disease-associated variants or in LD with known non-inflammatory disease-associated variants with r2>0.2 in the “1000 Genomes Project” phase3 data (available at www.internationalgenome.org/category/phase-3/, which is incorporated herein by reference in its entirety). The first number of variants that are either known or in LD (r2>0.2) with known non-inflammatory disease-associated variants may constitute the “DL-known” set of SNPs, and the remaining variants not in LD with known variants may constitute the “DL-others” set of SNPs.

Deep learning prediction model building may be performed as follows. A multi-layer feedforward artificial neural network, also known as a convolutional neural network (CNN), may be applied to the genetic datasets. The CNN may be a deep learning algorithm that is trained with a stochastic gradient descent using back-propagation. The network may contain a large number of hidden layers consisting of neurons with activation functions (e.g., tanh, rectifier, or maxout activation functions). Advanced features such as adaptive learning rate, rate annealing, momentum training, dropout, L₁ or L₂ regularization, checkpointing, and grid search may be used to enable high predictive accuracy. Further, the prediction model may be further developed by integration with other machine learning approaches, such as XGBoost, Gradient Boost, and Random Forest, to further improve the prediction performance. In addition, the incorporation of other “-omics” data (e.g., transcriptome and microbiome) may enable more informative predictions. Further, the methods and systems disclosed herein may be applied to develop prediction models for a variety of complex diseases, including non-inflammatory diseases, cardiovascular disease (CVD), adolescent idiopathic scoliosis, and type-2 diabetes (T2D).

In some embodiments, a CNN may be a tuple N=(L, T, Φ), where each of its elements is defined as follows: L=L₁₋ₖ is a set of layers such that L₁ is the input layer, L_{K} is the output layer, and the layers other than the input layer and the output layer are called hidden layers. Each layer Lₖ may comprise sₖ nodes, which are also called neurons. The l-th neuron of layer k may be denoted by nₖ. T⊆L×L may be a set of connections between layers such that, except for the input and output layers, each layer has an incoming connection and an outgoing connection. Φ=φ₁₋ₖ may be a set of activation functions φₖ: one for each non-input layer. The value of n_{k,l} may be denoted by v_{k,l}. Except for the input nodes, every node may be connected to nodes in the preceding layer by pre-defined weights for all k and l with 2≤k≤K and 1≤l≤sₖ. Finally, for any input, the neural network may assign a label, that is, the index of the node of output layer with the largest value: label=argmax_{1≤l≤sK}.

The deep learning algorithm may be applied separately to the first number of variants that are either known or in LD (r2>0.2) with known non-inflammatory disease-associated variants (DL-known), and the remaining variants not in LD with known variants (DL-others). A 5-fold cross-validation may be applied to control for model overfitting, and an ensemble model (DL-all) based on Support Vector Machine (SVM) may be built to combine DL-known and DL-others, again with 5-fold cross-validation. After building up different deep learning models in the training dataset, those models may be fitted in the test dataset to obtain the predictions. Deep learning analysis may be performed in the software H2O, and grid search may be performed to determine the best parameter settings for DL-known and DL-others. LDpred prediction may be performed as follows. LDpred analysis may be performed using the default parameters, based on the summary statistics from the non-inflammatory disease cohort. The calculated prediction score may be transformed into a probability using a logit transformation. The LDpred package in Python may be used for this analysis.

Evaluation of prediction performance may be performed as follows. Receiver Operating Characteristic (ROC) curves may be generated for different prediction models in the test dataset, and Area Under Curve (AUC) values may be calculated from the ROC curves and compared, such as by using the R package pROC. Also, the performance of difference approaches may be evaluated in enrichment of non-inflammatory disease cases in the extreme of non-inflammatory disease risk prediction. All these comparisons may be performed in the R software package.

High-order combination analysis may be performed as follows. As a preliminary step to explore the effects of non-linear effects in known variants, the combination effects of variants used in DL-known analysis may be examined using LAMPlink software (as described by, for example, Terada et al., “LAMPLINK: detection of statistically significant SNP combinations from GWAS data”, Bioinformatics, 32(22), 2016, 3513-3515, which is incorporated herein by reference in its entirety). Combinations of both dominant and recessive models may be performed, and LD filtering with an r2 cutoff of 0.2 may be performed to exclude potential contamination from SNPs in strong LD with each other.

Association of predicted risk with clinical phenotypes may be performed as follows. Association of prediction score from different algorithms with clinical characteristics may be evaluated in the generalized linear model framework, with Principal Components from population stratification analysis included as covariates.

Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module may comprise computer systems that connect laboratory hardware or instrumentation with computer systems that process laboratory data. In one embodiment, the data pre-processing module may comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to an non-inflammatory disease, pathology, state, risk, condition, or phenotype. A data interpretation module may use analysis methods, for example, drawn from statistics, mathematics, or biology, to support understanding of the relation between the identified abnormal patterns and health conditions, functional states, prognoses, or risks. A data visualization module may use methods of mathematical modeling, computer graphics, or rendering to create visual representations of data that may facilitate the understanding or interpretation of results.

Feature sets may be generated from datasets obtained using one or more assays of a biological sample, and a DeepLearning algorithm may be used to process one or more of the feature sets to identify or assess the non-inflammatory disease or condition. For example, the DeepLearning algorithm may be used to apply a machine learning classifier to a plurality of non-inflammatory disease-associated genomic loci that are associated with two or more classes of individuals inputted into a machine learning model, in order to classify a subject into one of the two or more classes of individuals. For example, the DeepLearning algorithm may be used to apply a machine learning classifier to a plurality of non-inflammatory disease-associated genomic loci that are associated with individuals with known conditions (e.g., an non-inflammatory disease or disorder) and individuals not having the condition (e.g., healthy individuals, or individuals who do not have an non-inflammatory disease or disorder), in order to classify a subject as having the condition (e.g., positive test outcome) or not having the condition (e.g., negative test outcome).

The DeepLearning algorithm may be configured to identify the presence (e.g., positive test result) or absence (e.g., negative test result) of one or more non-inflammatory disease or conditions with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The DeepLearning algorithm may comprise a machine learning algorithm, such as a supervised machine learning algorithm. The supervised machine learning algorithm may comprise, for example, a Random Forest, a support vector machine (SVM), a neural network, or a deep learning algorithm. The DeepLearning algorithm may comprise a classification and regression tree (CART) algorithm. The DeepLearning algorithm may comprise an unsupervised machine learning algorithm.

The DeepLearning algorithm may comprise a classifier configured to accept as input a plurality of input variables or features (e.g., non-inflammatory disease-associated genomic loci) and to produce or output one or more output values based on the plurality of input variables or features (e.g., non-inflammatory disease-associated genomic loci). The plurality of input variables or features may comprise one or more datasets indicative of the presence (e.g., positive test result) or absence (e.g., negative test result) of one or more non-inflammatory disease or conditions. For example, an input variable or feature may comprise a number of sequences corresponding to or aligning to each of the plurality of non-inflammatory disease-associated genomic loci.

The plurality of input variables or features may also include clinical information of a subject, such as health data. For example, the health data of a subject may comprise one or more of: a diagnosis of one or more non-inflammatory disease or conditions, a prognosis of one or more non-inflammatory disease or conditions, a risk of having one or more non-inflammatory disease or conditions, screening or testing results of one of more non-inflammatory disease or conditions, a treatment history of one or more non-inflammatory disease or conditions, a history of previous treatment for one or more non-inflammatory disease or conditions, a history of prescribed or other medications, a history of prescribed medical devices, personal characteristics (e.g., age, race, ethnicity, height, weight, sex, geographic location, diet, exercise, smoking status, family history of IBD), and one or more symptoms of the subject.

For example, the non-inflammatory disease or condition may comprise one or more of: cardiovascular disease, adolescent idiopathic scoliosis, a neurological disease, a fibrotic disease (e.g., PSC, scleroderma, or pulmonary fibrosis), type 2 diabetes, Alzheimer's disease, and obesity. As another example, the symptoms may include one or more of: pain, fatigue, nausea, weight loss, weakness, bleeding, loss of function, or a combination thereof. As another example, the screening or testing results may include one or more of: a blood test, X-ray scan, computerized tomography (CT) scan, positron emission tomography (PET) scan, PET-CT scan, magnetic resonance imaging (MRI), ultrasound scan, or a combination thereof. As another example, the prescribed or other medications or drugs may include one or more of: drugs, antibiotics, anti-diarrheal medications, pain relievers, iron supplements, calcium supplements, vitamin D supplements, or a combination thereof. As another example, the previous treatment for non-inflammatory disease or conditions may include surgery.

Table 1 shows an example of non-inflammatory diseases and associated training cohorts from which non-inflammatory diseases and/or fibrotic cases and/or controls may be obtained.

TABLE 1
Non-inflammatory or Fibrotic Diseases and Associated Training Cohorts

| Disease | Training Cohort | Sample size |
|---|---|---|
| Type 2 Diabetes | Framingham Cohort, | 213,396 |
| | Women's Health Study, | |
| | eMerge Network | |
| Alzheimer’s Disease | ADGC,GENADA,NIA-late | 12,228 |
| Obesity | Framingham Cohort, | 213,396 |
| | Women's Health Study, | |
| | eMerge Network | |
| Cardiovascular Disease | UKB dataset | 500,000 |
| | AIS | 150,000 |
| Primary sclerosing | UK-PSC Consortium, The | 4,796 (cases) |
| cholangitis (PSC) | International IBD Genetics | 19,955 (controls) |
| | Consortium, The | |
| | International PSC Study | |
| | Group | |
| Scleroderma | European Scleroderma | 8231 (cases) |
| | Group and Australia | 10356 (controls) |
| | Scleroderma Group | |
| Pulmonary Fibrosis | IPF case-control collections | 3668 (cases) |
| | | 17,951 (controls) |

The DeepLearning algorithm may comprise a classifier, such that each of the one or more output values comprises one of a fixed number of possible values (e.g., a linear classifier, a logistic regression classifier, etc.) indicating a classification of the sample by the classifier. The DeepLearning algorithm may comprise a binary classifier, such that each of the one or more output values comprises one of two values (e.g., {0, 1}, {positive, negative}, or {high-risk, low-risk}) indicating a classification of the sample by the classifier. The DeepLearning algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more non-inflammatory disease or conditions of the subject, and may comprise, for example, positive, negative, high-risk, intermediate-risk, low-risk, or indeterminate. Such descriptive labels may provide an identification of a treatment for the one or more non-inflammatory disease or conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, blood test, X-ray scan, computerized tomography (CT) scan, positron emission tomography (PET) scan, PET-CT scan, magnetic resonance imaging (MRI), ultrasound scan, or a combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more non-inflammatory disease or conditions of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more non-inflammatory disease or conditions, thereby assigning the subject to a class of individuals receiving a positive test result. As another example, a binary classification of samples may assign an output value of “negative” or 0 if the sample indicates that the subject has less than a 50% probability of having one or more non-inflammatory disease or conditions, thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more non-inflammatory disease or conditions of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more non-inflammatory disease or conditions of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more non-inflammatory disease or conditions of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more non-inflammatory disease or conditions of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more non-inflammatory disease or conditions, such as an non-inflammatory disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The DeepLearning algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of an non-inflammatory disease or condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more non-inflammatory disease or conditions of the subject. Independent training samples may be associated with presence of the non-inflammatory disease or condition (e.g., training samples comprising samples and associated datasets and outputs obtained or derived from a plurality of subjects known to have the non-inflammatory disease or condition). Independent training samples may be associated with absence of the non-inflammatory disease or condition (e.g., training samples comprising samples and associated datasets and outputs obtained or derived from a plurality of subjects who are known to not have a previous diagnosis of the non-inflammatory disease or condition or who have received a negative test result for the non-inflammatory disease or condition).

The DeepLearning algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the non-inflammatory disease or condition. The DeepLearning algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the non-inflammatory disease or condition. The DeepLearning algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the non-inflammatory disease or condition. In some embodiments, the sample is independent of samples used to train the DeepLearning algorithm.

The DeepLearning algorithm may be trained with a first number of independent training samples associated with a presence of the non-inflammatory disease or condition and a second number of independent training samples associated with an absence of the non-inflammatory disease or condition. The first number of independent training samples associated with presence of the non-inflammatory disease or condition may be no more than the second number of independent training samples associated with absence of the non-inflammatory disease or condition. The first number of independent training samples associated with a presence of the non-inflammatory disease or condition may be equal to the second number of independent training samples associated with an absence of the non-inflammatory disease or condition. The first number of independent training samples associated with a presence of the non-inflammatory disease or condition may be greater than the second number of independent training samples associated with an absence of the non-inflammatory disease or condition.

The DeepLearning algorithm may comprise a classifier configured to identify the presence (e.g., positive test result) or absence (e.g., negative test result) of one or more non-inflammatory disease or conditions at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the DeepLearning algorithm may be calculated as the percentage of independent test samples (e.g., subjects known to have the non-inflammatory disease or condition or subjects with negative clinical test results for the non-inflammatory disease or condition) that are correctly identified or classified as having or not having the non-inflammatory disease or condition.

The DeepLearning algorithm may comprise a classifier configured to identify one or more non-inflammatory diseases or conditions with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the non-inflammatory disease or condition using the DeepLearning algorithm may be calculated as the percentage of samples identified or classified as having the non-inflammatory disease or condition that correspond to subjects that truly have the non-inflammatory disease or condition.

The DeepLearning algorithm may comprise a classifier configured to identify one or more non-inflammatory disease or conditions with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the non-inflammatory disease or condition using the DeepLearning algorithm may be calculated as the percentage of samples identified or classified as not having the non-inflammatory disease or condition that correspond to subjects that truly do not have the non-inflammatory disease or condition.

The DeepLearning algorithm may comprise a classifier configured to identify one or more non-inflammatory disease or conditions with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the non-inflammatory disease or condition using the DeepLearning algorithm may be calculated as the percentage of independent test samples associated with presence of the non-inflammatory disease or condition (e.g., subjects known to have the non-inflammatory disease or condition) that are correctly identified or classified as having the non-inflammatory disease or condition.

The DeepLearning algorithm may comprise a classifier configured to identify one or more non-inflammatory disease or conditions with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the non-inflammatory disease or condition using the DeepLearning algorithm may be calculated as the percentage of independent test samples associated with absence of the non-inflammatory disease or condition (e.g., subjects with negative clinical test results for the non-inflammatory disease or condition) that are correctly identified or classified as not having the non-inflammatory disease or condition.

The DeepLearning algorithm may comprise a classifier configured to identify the presence (e.g., positive test result) or absence (e.g., negative test result) of one or more non-inflammatory disease or conditions with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the DeepLearning algorithm in classifying samples as having or not having the non-inflammatory disease or condition. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

Classifiers of the DeepLearning algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the non-inflammatory disease or condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The DeepLearning algorithm may comprise a plurality of classifiers (e.g., an ensemble) such that the plurality of classifications or outcome values of the plurality of classifiers may be combined to produce a single classification or outcome value for the sample (e.g., to generate an ensemble output). For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the DeepLearning algorithm is initially trained, a subset of the inputs may be identified as most influential or most important to be included for making high-quality classifications (e.g., having highest permutation feature importance). For example, a subset of the panel of non-inflammatory disease-associated genomic loci may be identified as most influential or most important to be included for making high-quality classifications or identifications of non-inflammatory disease or conditions (or sub-types of non-inflammatory disease or conditions). The panel of non-inflammatory disease-associated genomic loci, or a subset thereof, may be ranked based on classification metrics indicative of each influence or importance of each individual non-inflammatory disease-associated genomic locus toward making high-quality classifications or identifications of non-inflammatory disease or conditions (or sub-types of non-inflammatory disease or conditions). Such metrics may be used to reduce, in some cases significantly, the number of input variables (e.g., predictor variables) that may be used to train the one or more classifiers of the DeepLearning algorithm to a desired performance level (e.g., based on a desired minimum accuracy, PPV, NPV, clinical sensitivity, clinical specificity, AUC, or a combination thereof).

For example, if training a classifier of the DeepLearning algorithm with a plurality comprising several dozen or hundreds of input variables to the classifier results in an accuracy of classification of more than 99%, then training the classifier of the DeepLearning algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the DeepLearning algorithm with a plurality comprising several dozen or hundreds of input variables to the classifier results in a sensitivity or specificity of classification of more than 99%, then training the classifier of the DeepLearning algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of non-inflammatory disease-associated genomic loci) to the classifier of the DeepLearning algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more non-inflammatory disease or conditions, the subject may be optionally provided with a therapeutic intervention (e.g., prescribing an appropriate course of treatment to treat the one or more non-inflammatory disease or conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

In some embodiments, a DeepLearning model may be used to predict a level of efficacy (e.g., a response or a non-response) of a given therapeutic intervention for an non-inflammatory disease of a subject. In some embodiments, a therapeutic intervention may be selected from one or more therapeutic interventions based on maximizing a predicted level of efficacy of the therapeutic intervention, minimizing side effects of the therapeutic intervention, minimizing a cost of the therapeutic intervention, or a combination thereof.

In some embodiments, upon identifying a subject as having elevated risk of developing an non-inflammatory disease with the DeepLeaning model described herein, a primary intervention may be administered to the subject to prevent or delay the onset of the non-inflammatory disease or condition. For example, a primary intervention may effectively delay onset of rheumatoid arthritis in a subject having elevated or high risk thereof.

The therapeutic intervention may include prescribed or other medications or drugs, which may include one or more of: anti-non-inflammatory drugs, immunosuppressant drugs, antibiotics, anti-diarrheal medications, pain relievers, iron supplements, calcium supplements, vitamin D supplements, or a combination thereof. The therapeutic intervention may include surgery (e.g., colectomy). The therapeutic intervention may be effective to alleviate or decrease one or more symptoms, which may include one or more of: pain, fatigue, nausea, weight loss, weakness, bleeding, loss of function, or a combination thereof.

The therapeutic intervention may comprise recommending the subject for a secondary clinical test to confirm a diagnosis of the non-inflammatory disease or condition. This secondary clinical test may comprise a blood test, X-ray scan, computerized tomography (CT) scan, positron emission tomography (PET) scan, PET-CT scan, magnetic resonance imaging (MRI), ultrasound scan, or a combination thereof.

The feature sets (e.g., comprising quantitative measures of a panel of non-inflammatory disease-associated genomic loci) may be analyzed and assessed (e.g., using a DeepLearning algorithm comprising one or more classifiers) over a duration of time to monitor a patient (e.g., subject who has an non-inflammatory disease or condition or who is being treated for an non-inflammatory disease or condition). In such cases, the feature sets of the patient may change during the course of treatment. For example, the quantitative measures of the feature sets of a patient with decreasing risk of the non-inflammatory disease or condition due to an effective treatment may shift toward the profile or distribution of a healthy subject (e.g., a subject without the non-inflammatory disease or condition). Conversely, for example, the quantitative measures of the feature sets of a patient with increasing risk of the non-inflammatory disease or condition due to an ineffective treatment may shift toward the profile or distribution of a subject with higher risk of the non-inflammatory disease or condition or a more advanced stage or severity of the non-inflammatory disease or condition.

The non-inflammatory disease or condition of the subject may be monitored by monitoring a course of treatment for treating the non-inflammatory disease or condition of the subject. The monitoring may comprise assessing the non-inflammatory disease or condition of the subject at two or more time points. The assessing may be based at least on the feature sets (e.g., quantitative measures of a panel of non-inflammatory disease-associated genomic loci) determined at each of the two or more time points. The therapeutic intervention may include prescribed or other medications or drugs, which may include one or more of: drugs, antibiotics, anti-diarrheal medications, pain relievers, iron supplements, calcium supplements, vitamin D supplements, or a combination thereof. The therapeutic intervention may include surgery. The therapeutic intervention may be effective to alleviate or decrease one or more symptoms, which may include one or more of: pain, fatigue, nausea, weight loss, weakness, bleeding, loss of function, or a combination thereof. The assessing may be based at least on the presence, absence, or severity of one or more symptoms, such as pain, fatigue, nausea, weight loss, weakness, bleeding, loss of function, or a combination thereof.

In some embodiments, a difference in the feature sets (e.g., quantitative measures of a panel of non-inflammatory disease-associated genomic loci) determined between the two or more time points may be indicative of one or more clinical indications, such as (i) a diagnosis of the non-inflammatory disease or condition of the subject, (ii) a prognosis of the non-inflammatory disease or condition of the subject, (iii) an increased risk of the non-inflammatory disease or condition of the subject, (iv) a decreased risk of the non-inflammatory disease or condition of the subject, (v) an efficacy of the course of treatment for treating the non-inflammatory disease or condition of the subject, and (vi) a non-efficacy of the course of treatment for treating the non-inflammatory disease or condition of the subject.

In some embodiments, a difference in the feature sets (e.g., quantitative measures of a panel of non-inflammatory disease-associated genomic loci) determined between the two or more time points may be indicative of a diagnosis of the non-inflammatory disease or condition of the subject. For example, if the non-inflammatory disease or condition was not detected in the subject at an earlier time point but was detected in the subject at a later time point, then the difference is indicative of a diagnosis of the non-inflammatory disease or condition of the subject. A clinical action or decision may be made based on this indication of diagnosis of the non-inflammatory disease or condition of the subject, such as, for example, prescribing a new therapeutic intervention for the subject. The clinical action or decision may comprise recommending the subject for a secondary clinical test to confirm the diagnosis of the condition. This secondary clinical test may include one or more of: a blood test, X-ray scan, computerized tomography (CT) scan, positron emission tomography (PET) scan, PET-CT scan, magnetic resonance imaging (MRI), ultrasound scan, or a combination thereof.

In some embodiments, a difference in the feature sets (e.g., quantitative measures of a panel of non-inflammatory disease-associated genomic loci) determined between the two or more time points may be indicative of a prognosis of the non-inflammatory disease or condition of the subject.

In some embodiments, a difference in the feature sets (e.g., quantitative measures of a panel of non-inflammatory disease-associated genomic loci) determined between the two or more time points may be indicative of the subject having an increased risk of the non-inflammatory disease or condition. For example, if the non-inflammatory disease or condition was detected in the subject both at an earlier time point and at a later time point, and if the quantitative measures of a panel of non-inflammatory disease-associated genomic loci increased from the earlier time point to the later time point, then the difference may be indicative of the subject having an increased risk of the non-inflammatory disease or condition. A clinical action or decision may be made based on this indication of the increased risk of the non-inflammatory disease or condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may include one or more of: a blood test, X-ray scan, computerized tomography (CT) scan, positron emission tomography (PET) scan, PET-CT scan, magnetic resonance imaging (MRI), ultrasound scan, or a combination thereof.

In some embodiments, a difference in the feature sets (e.g., quantitative measures of a panel of non-inflammatory disease-associated genomic loci) determined between the two or more time points may be indicative of the subject having a decreased risk of the non-inflammatory disease or condition. For example, if the non-inflammatory disease or condition was detected in the subject both at an earlier time point and at a later time point, and if the quantitative measures of a panel of non-inflammatory disease-associated genomic loci decreased from the earlier time point to the later time point, then the difference may be indicative of the subject having a decreased risk of the non-inflammatory disease or condition. A clinical action or decision may be made based on this indication of the decreased risk of the non-inflammatory disease or condition (e.g., continuing or ending a current therapeutic intervention) for the subject. The clinical action or decision may comprise recommending the subject for a secondary clinical test to confirm the decreased risk of the condition. This secondary clinical test may include one or more of: a blood test, X-ray scan, computerized tomography (CT) scan, positron emission tomography (PET) scan, PET-CT scan, magnetic resonance imaging (MRI), ultrasound scan, or a combination thereof.

In some embodiments, a difference in the feature sets (e.g., quantitative measures of a panel of non-inflammatory disease-associated genomic loci) determined between the two or more time points may be indicative of an efficacy of the course of treatment for treating the non-inflammatory disease or condition of the subject. For example, if the non-inflammatory disease or condition was detected in the subject at an earlier time point but was not detected in the subject at a later time point, then the difference may be indicative of an efficacy of the course of treatment for treating the non-inflammatory disease or condition of the subject. A clinical action or decision may be made based on this indication of the efficacy of the course of treatment for treating the non-inflammatory disease or condition of the subject, e.g., continuing or ending a current therapeutic intervention for the subject. The clinical action or decision may comprise recommending the subject for a secondary clinical test to confirm the efficacy of the course of treatment for treating the non-inflammatory disease or condition. This secondary clinical test may include one or more of: a blood test, X-ray scan, computerized tomography (CT) scan, positron emission tomography (PET) scan, PET-CT scan, magnetic resonance imaging (MRI), ultrasound scan, or a combination thereof.

In some embodiments, a difference in the feature sets (e.g., quantitative measures of a panel of non-inflammatory disease-associated genomic loci) determined between the two or more time points may be indicative of a non-efficacy of the course of treatment for treating the non-inflammatory disease or condition of the subject. For example, if the non-inflammatory disease or condition was detected in the subject both at an earlier time point and at a later time point, and if the quantitative measures of a panel of non-inflammatory disease-associated genomic loci increased or remained at a constant level from the earlier time point to the later time point, and if an efficacious treatment was indicated at an earlier time point, then the difference may be indicative of a non-efficacy of the course of treatment for treating the non-inflammatory disease or condition of the subject. A clinical action or decision may be made based on this indication of the non-efficacy of the course of treatment for treating the non-inflammatory disease or condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the non-inflammatory disease or condition. This secondary clinical test may include one or more of: a blood test, X-ray scan, computerized tomography (CT) scan, positron emission tomography (PET) scan, PET-CT scan, magnetic resonance imaging (MRI), ultrasound scan, or a combination thereof.

In various embodiments, machine learning methods are applied to distinguish samples in a population of samples. In one embodiment, machine learning methods are applied to distinguish samples between healthy and non-inflammatory disease samples.

### III. KITS

The present disclosure provides kits for identifying or monitoring an non-inflammatory disease or condition of a subject. A kit may comprise probes for identifying a quantitative measure (e.g., indicative of a presence, absence, or relative amount) of sequences at each of a panel of non-inflammatory disease-associated genomic loci in a sample of the subject. A quantitative measure (e.g., indicative of a presence, absence, or relative amount) of sequences at each of a panel of non-inflammatory disease-associated genomic loci in the sample may be indicative of the non-inflammatory disease or condition of the subject. The probes may be selective for the sequences at the panel of non-inflammatory disease-associated genomic loci in the sample. A kit may comprise instructions for using the probes to process the sample to generate datasets indicative of a quantitative measure (e.g., indicative of a presence, absence, or relative amount) of sequences at each of the panel of non-inflammatory disease-associated genomic loci in a sample of the subject.

The probes in the kit may be selective for the sequences at the panel of non-inflammatory disease-associated genomic loci in the sample. The probes in the kit may be configured to selectively enrich nucleic acid (e.g., RNA or DNA) molecules corresponding to the panel of non-inflammatory disease-associated genomic loci. For example, the non-inflammatory disease-associated genomic loci may be associated with one or more single nucleotide polymorphisms (SNPs), copy number variations (CNVs), insertions or deletions (indels), fusions, translocations, or other genetic variants. The probes may be nucleic acid primers. The probes may have sequence complementarity with nucleic acid sequences from one or more of the panel of non-inflammatory disease-associated genomic loci. The panel of non-inflammatory disease-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more non-inflammatory disease-associated genomic loci.

The instructions in the kit may comprise instructions to assay the sample using the probes that are selective for the sequences at the panel of non-inflammatory disease-associated genomic loci in the sample. These probes may be nucleic acid molecules (e.g., RNA or DNA) having sequence complementarity with nucleic acid sequences (e.g., RNA or DNA) from one or more of the plurality of panel of non-inflammatory disease-associated genomic loci. These nucleic acid molecules may be primers or enrichment sequences. The instructions to assay the sample may comprise instructions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of non-inflammatory disease-associated genomic loci in the sample. A quantitative measure (e.g., indicative of a presence, absence, or relative amount) of sequences at each of a panel of non-inflammatory disease-associated genomic loci in the sample may be indicative of an non-inflammatory disease or condition.

The instructions in the kit may comprise instructions to measure and interpret assay readouts, which may be quantified at one or more of the panel of non-inflammatory disease-associated genomic loci to generate the datasets indicative of a quantitative measure (e.g., indicative of a presence, absence, or relative amount) of sequences at each of the panel of non-inflammatory disease-associated genomic loci in the sample. For example, quantification of array hybridization or polymerase chain reaction (PCR) corresponding to the panel of non-inflammatory disease-associated genomic loci may generate the datasets indicative of a quantitative measure (e.g., indicative of a presence, absence, or relative amount) of sequences at each of the panel of non-inflammatory disease-associated genomic loci in the sample. Assay readouts may comprise quantitative PCR (qPCR) values, digital PCR (dPCR) values, digital droplet PCR (ddPCR) values, fluorescence values, etc., or normalized values thereof.

### IV. COMPUTER SYSTEMS

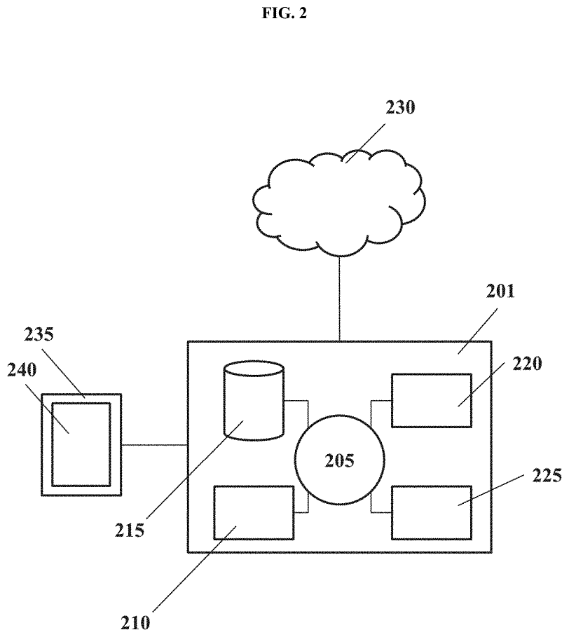

The present disclosure provides computer systems that are programmed to implement methods of the disclosure. FIG. 2 shows a computer system 201 that is programmed or otherwise configured to, for example, (i) train and test a DeepLearning algorithm, (ii) use the DeepLearning algorithm to process data to determine an non-inflammatory disease or condition of a subject, (iii) determine a quantitative measure indicative of an non-inflammatory disease or condition of a subject, (iv) identify or monitor the non-inflammatory disease or condition of the subject, and (v) electronically output a report that indicative of the non-inflammatory disease or condition of the subject.

The computer system 201 may regulate various aspects of analysis, calculation, and generation of the present disclosure, such as, for example, (i) training and testing a DeepLearning algorithm, (ii) using the DeepLearning algorithm to process data to determine an non-inflammatory disease or condition of a subject, (iii) determining a quantitative measure indicative of an non-inflammatory disease or condition of a subject, (iv) identifying or monitoring the non-inflammatory disease or condition of the subject, and (v) electronically outputting a report that indicative of the non-inflammatory disease or condition of the subject. The computer system 201 may be an electronic device of a user or a computer system that is remotely located with respect to the electronic device. The electronic device may be a mobile electronic device.

The computer system 201 includes a central processing unit (CPU, also “processor” and “computer processor” herein) 205, which may be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 may be a data storage unit (or data repository) for storing data. The computer system 201 may be operatively coupled to a computer network (“network”) 230 with the aid of the communication interface 220. The network 230 may be the Internet, an internet and/or extranet, or an intranet and/or extranet that is in communication with the Internet.

The network 230 in some cases is a telecommunication and/or data network. The network 230 may include one or more computer servers, which may enable distributed computing, such as cloud computing. For example, one or more computer servers may enable cloud computing over the network 230 (“the cloud”) to perform various aspects of analysis, calculation, and generation of the present disclosure, such as, for example, (i) training and testing a DeepLearning algorithm, (ii) using the DeepLearning algorithm to process data to determine an non-inflammatory disease or condition of a subject, (iii) determining a quantitative measure indicative of an non-inflammatory disease or condition of a subject, (iv) identifying or monitoring the non-inflammatory disease or condition of the subject, and (v) electronically outputting a report that indicative of the non-inflammatory disease or condition of the subject. Such cloud computing may be provided by cloud computing platforms such as, for example, Amazon Web Services (AWS), Microsoft Azure, Google Cloud Platform, and IBM cloud. The network 230, in some cases with the aid of the computer system 201, may implement a peer-to-peer network, which may enable devices coupled to the computer system 201 to behave as a client or a server.

The CPU 205 may comprise one or more computer processors and/or one or more graphics processing units (GPUs). The CPU 205 may execute a sequence of machine-readable instructions, which may be embodied in a program or software. The instructions may be stored in a memory location, such as the memory 210. The instructions may be directed to the CPU 205, which may subsequently program or otherwise configure the CPU 205 to implement methods of the present disclosure. Examples of operations performed by the CPU 205 may include fetch, decode, execute, and writeback.

The CPU 205 may be part of a circuit, such as an integrated circuit. One or more other components of the system 201 may be included in the circuit. In some cases, the circuit is an application specific integrated circuit (ASIC).

The storage unit 215 may store files, such as drivers, libraries and saved programs. The storage unit 215 may store user data, e.g., user preferences and user programs. The computer system 201 in some cases may include one or more additional data storage units that are external to the computer system 201, such as located on a remote server that is in communication with the computer system 201 through an intranet or the Internet.

The computer system 201 may communicate with one or more remote computer systems through the network 230. For instance, the computer system 201 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 201 via the network 230.

Methods as described herein may be implemented by way of machine (e.g., computer processor) executable code stored on an electronic storage location of the computer system 201, such as, for example, on the memory 210 or electronic storage unit 215. The machine-executable or machine-readable code may be provided in the form of software. During use, the code can be executed by the processor 205. In some cases, the code may be retrieved from the storage unit 215 and stored on the memory 210 for ready access by the processor 205. In some situations, the electronic storage unit 215 may be precluded, and machine-executable instructions are stored on memory 210.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 201, may be embodied in programming. Various aspects of the technology may be thought of as “products” or “articles of manufacture” typically in the form of machine (or processor) executable code and/or associated data that is carried on or embodied in a type of machine-readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 201 may include or be in communication with an electronic display 235 that comprises a user interface (UI) 240 for providing, for example, (i) a visual display indicative of training and testing of a DeepLearning algorithm, (ii) a visual display of data indicative of an non-inflammatory disease or condition of a subject, (iii) a quantitative measure of an non-inflammatory disease or condition of a subject, (iv) an identification of a subject as having an non-inflammatory disease or condition, or (v) an electronic report indicative of the non-inflammatory disease or condition of the subject. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 205. The algorithm can, for example, (i) train and test a DeepLearning algorithm, (ii) use the DeepLearning algorithm to process data to determine an non-inflammatory disease or condition of a subject, (iii) determine a quantitative measure indicative of an non-inflammatory disease or condition of a subject, (iv) identify or monitor the non-inflammatory disease or condition of the subject, and (v) electronically output a report that indicative of the non-inflammatory disease or condition of the subject.

### V. EXAMPLES

The following examples are included for illustrative purposes only and are not intended to limit the scope of the invention.

### Example 1: Using DeepLearning and Genetic BigData to Construct a Disease Prediction Model for Non-Inflammatory Disease

A Deep Learning (DL) model may be built, validated and tested to predict a non-inflammatory disease using genetic data. The performance of the DL model in this example is compared to the performance of LDpred. The DL model in this example and according to various embodiments described herein may yield more accurate predictions as compared to LDpred, underscoring the clinical utility of the DL model in clinical practice to inform decision-making (e.g., diagnosis, prognosis, selection of therapeutic intervention, disease and/or therapeutic regimen monitoring, and the like).

Methods: DL may be utilized to build a disease prediction model with a first number of patients with non-inflammatory disease and a second number of controls from a non-inflammatory disease cohort as the training dataset. This model may be further validated in non-inflammatory disease cases and non-disease controls that were independent from the training set. Both training and validation cohorts may be genotyped using ImmunoChip. A set of SNPs that may be successfully measured in both cohorts and pass the stringent QC are included as predictors. A convolutional neural network (CNN) algorithm may be used to construct a DL model, and cross-validation may be performed as part of the DL model construction. Further, the association of the DL prediction score may be examined with clinical phenotypes.

Performance of the DL model may be compared to the LDpred algorithm (e.g., as described by Amit V. Khera et. al, Nature Genetics, 2018; which is incorporated herein by reference in its entirety). A non-trivial improvement in prediction performance of DL may be observed with a greater Area Under the Curve (AUC) as compared to that using LDPred. The predicted risk from DL may lead to greatly enriched cases in the extreme of the DL score, as indicated by the OR. Utilizing only known non-inflammatory disease-susceptibility variants (and variants in LD with known), the DL based algorithm (DL-known) may achieve a high AUC. Further analyses may indicate that the improved performance of the DL-known score is likely through its ability to incorporate non-linear causal effects. Moreover, after excluding known variants, a high AUC may be observed with the DL algorithm (DL-other). Variance importance metrics of the DL-other algorithm may identify a number of novel non-inflammatory disease variants that achieve genome-wide significance in a meta-analysis incorporating a large set of thousands of individuals. DL predicted risk score may also be strongly associated with non-inflammatory disease clinical phenotypes including disease location, severity, and need for surgery. Therefore, utilizing this genetic algorithm, individuals with monogenic-like disease risk for the non-inflammatory disease may be identified, a capability that provides progress towards early diagnosis and identifying subjects for studying preventative strategies.

The Deep Learning prediction models may be constructed as follows. A multi-layer feedforward artificial neural network, also known as convolutional neural network (CNN), may be utilized to build the prediction model. The CNN model may be constructed separately with a) the first number of variants that are either known or in LD (r2>0.2) with known non-inflammatory disease variants (DL-known), and b) the remaining variants not in LD with known variants (DL-others). The CNN model may be optimized in the software H2O using stochastic gradient descent with both L₁ and L₂ regularization. A grid search may be performed to determine the best parameter settings separately for DL-known and DL-others, including numbers of hidden layers, number of neurons in each layer, activation functions of the layers, dropout ratio, and parameters for L₁ and L₂ regularization. In the trained models, the variable relative importance may be calculated using Gedeon's approach, based on the weights connecting the input features to the first two hidden layers. A 5-fold cross-validation may be applied to the control for model overfitting. Further, an ensemble model (DL-comb) based on Support Vector Machine (SVM) may be built to combine DL-known and DL-others with 5-fold cross-validation. After building up different Deep Learning models in the training dataset, models may be fitted using the test datasets. The final prediction model may be used as a non-inflammatory disease risk prediction tool.

Prediction performance of the deep learning algorithm may be compared to the LDPred approach as follows.

LDpred analysis may be performed using the default parameters, based on the summary statistics from the non-inflammatory disease cohort. The LDPred23 Python package may be used for these analyses. LDPred analysis may be performed across different p-value thresholds (1.0E-6, 3.0E-6, 1.0E-5, 3.0E-5, 1.0E-4, 3.0E-4, 0.001, 0.003, 0.01, 0.05, 0.10, and 0.25), and the p-value threshold with best AUC may be selected.

Prediction performance may be evaluated as follows. Receiver Operating Characteristic (ROC) curves may be generated for different prediction models in the test dataset. Further, Area Under Curve (AUC) may be calculated for each of the ROC curves, and compared using the R package pROC31. Further, the performance of difference approaches may be evaluated in enrichment of non-inflammatory disease cases in the extreme of non-inflammatory disease risk prediction. All comparisons may be performed in the R software package.

High-order combination analysis may be performed as follows. To investigate the effects of non-linear effects in known variants (and variants associated with known), the combined effects of variants used in DL-known analysis may be examined using LAMPlink software. Combinations of both dominant and recessive models may be performed, and LD filtering may be performed with r2 cutoff of 0.2 to exclude potential contamination from SNPs in strong LD.

Association of single variants with non-inflammatory disease and meta-analysis may be performed as follows. Association of SNPs within the top 500 of variable importance with the non-inflammatory disease may be examined in the non-inflammatory disease cohorts separately, using logistic regression with adjustment for principal components from population stratification analysis. A meta-analysis may be performed to combine the summary statistics in both cohorts, after excluding overlapping samples.

Association of predicted risk with clinical phenotypes may be performed as follows. Association of prediction score from different algorithms with clinical characteristics may be evaluated in the generalized linear model framework, with Principal Components from population stratification analysis included as covariates.

After performing an intensive grid search to tune or optimize the hyperparameters, a CNN model with a suitable number of hidden layers (with a suitable number of neurons in each layer, with a suitable L₁ penalty and a suitable L₂ penalty) may be constructed for DL-known. For DL-others, a model with a suitable number of hidden layers (with a suitable number of neurons in each layer) with a suitable L₁ penalty and a suitable L₂ penalty may be constructed. A SVM model combining DL-known and DL-others may be then trained in the training cohort combining the DL-known and DL-others models.

A significant improvement in prediction performance may be observed using the deep learning algorithm compared to LDpred. Receiver operating characteristic (ROC) curves may be generated of different polygenic risk scores for DL_known (Deep Learning model using known susceptibility variants and variants in LD with susceptibility variants); DL_others (Deep Learning model using the other variants (e.g., excluding known susceptibility variants and variants in LD with these susceptibility variants)) on ImmunoChip™; DL_comb (Deep Learning model combining DL-known and DL-others). In the test set, the Area Under the Curve (AUC) of the LDpred approach (with p-value cutoff of 0.01) may be determined, while deep learning constructed using the known variants and variants in LD with known (DL-known) may exhibit an AUC which may be significantly higher than that of LDpred to a statistically significant extent. Deep learning with other variants (DL-others), where variants included in the DL-known analysis may be excluded, may exhibit an AUC which may be also higher than the LDpred prediction to a statistically significant extent. Combining the DL-known and DL-other variants (DL-comb) may improve the overall AUC of prediction to a statistically significant extent as compared to compared to LDpred prediction and as compared to DL-known), which may be among the best performance of risk prediction of complex human diseases using genetic data.

This improvement in prediction accuracy may lead to greatly enriched cases in both the extreme and even the not-so-extreme tails of the DL-comb scores. All deep-learning based approaches, whether based on the known variants, the remaining of the immunoChip, or the combined, may demonstrate better performance as compared to LDpred. For example, in the top 5% of the predicted risk, a higher Odds Ratio (OR) may be observed for DL-others, for DL-known, and for DL-comb, compared to the rest of the 95% of the samples (as compared to for LDpred). As another example, in the top 10% of the predicted risk, observed Odds Ratios (OR) may be higher for DL-others, for DL-known, and for DL-comb, compared to for LDpred. Within the top 5% and 10% of the DL-comb score, about 90% may be non-inflammatory disease patients. As a comparison, in LDPred algorithm, the proportion of non-inflammatory disease patients may be significantly lower in the top 5% and 10%. The corresponding positive likelihood ratio (LR+) may be greater for DL-comb using top 5% and 10% cutoff, as compared to for LDpred. And the corresponding negative likelihood ratio (LR−) may be lower for DL-comb, as compared to for LDPred. These all may indicate that the deep learning algorithms may greatly boost the practical potentials of genomic prediction.

Importantly, the DL model may be trained using the non-inflammatory disease cohort, in which most of the non-inflammatory disease patients may be adult. Further, the performance of the DL algorithm may be evaluated in another cohort, which may be a pediatric non-inflammatory disease cohort with ages of diagnosis of patients less than 16 years old. Similar performance of the DL algorithm may be observed in the pediatric non-inflammatory disease study, thereby confirming the DL model's robustness in this independent and heterogenous test cohort.

Interestingly, when examining the extremes of the deep learning scores, the ORs of DL-known and DL-comb may be comparable. This may indicate that with smartly constructed algorithms, screening for individuals with high disease risk may potentially be flexible. It may be performed based on the overall genetic profile across the genome (and potentially with best prediction accuracy), or alternatively only using a relatively small panel of known variants for a more flexible solution with slightly reduced accuracy. Again, this may make genomic prediction more practical and may have broad clinical impacts, potentially easing the path to clinical translation.

While the DL-others algorithm, only with variants not in LD with known variants, has an AUC which is less than those of the DL-known and DL-comb models, this still may represent an improvement over LDpred, which may indicate that there may be additional variants, probably with weak effects, contributing to the development of this complex disease. This may not be surprising given the “missing heritability” in non-inflammatory diseases and many other complex human traits, and perhaps the study of hundreds of thousands of individuals may be performed to identify the additional individual susceptibility variants. Deep learning score approaches may provide an alternative way to ‘collapse’ those variants to generate meaningful information with currently limited sample sizes. The DL algorithm may also indicate the contribution of each variant to the predicted disease risk score based on the variance importance metrics, which may be viewed as an indication of potential novel genetic loci.

The variance importance metrics of the DL-other model in non-inflammatory disease prediction may be examined. The variable importance metrics from the DL algorithm may indicate the relative importance or contribution of each variable and/or mutation to the overall model, which may be helpful in discovery of novel signals. For the top 500 variants, a meta-analysis may be performed incorporating the immunoChip data from the non-inflammatory disease cohorts. Of those 500 variants, a number of novel genome-wide signals may be identified with a meta-analysis p-value that is statistically significant.

Several interesting novel variants may be identified that are functionally relevant. For example, genes are identified may be implicated in the non-inflammatory disease or its pathogenesis, with a high, statistically significant OR and a high relative importance. In addition to predicting complex disease risk, the LD model may be a useful tool in identifying new genetic variants with functional relevance to complex disease pathology.

Within non-inflammatory disease cases in the validation cohort, the predicted DL score may be strongly related to clinical phenotypes, based on observed OR in severe or advanced-stage disease vs. mild or early-stage disease. DL score is also strongly associated with disease markers.

Further, the DL-known score may be plotted against the LDPred score in non-inflammatory disease patients. As may be expected in both cases of non-linear effects, all carriers of certain three SNP combination may be in the top-left side of the diagonal with higher estimated risk in DL-known compared to LDPred.

Conclusion: With this DL model, individuals with disease risk for an non-inflammatory disease may be identified using only genetic data, making it a powerful tool for disease early diagnosis and intervention. In some embodiments, the disease is monogenic-like. The utility of the DL model is not limited to predicting complex disease risk for non-inflammatory disease. Without being bound by any particular theory, this DL model may be utilized with a wide range of genetic data input (e.g., genetic variants associated with other non-inflammatory diseases or disorders) to predict complex disease risk.

### Example 2: Performance of the DL Algorithm and LDpred Approach in a Pediatric Cohort

Further, the trained model may be applied in a pediatric cohort. The pediatric cohort may be recruited according to an ongoing, prospective observational multi-center collaborative study of pediatric non-inflammatory disease. Children and adolescents younger than 17 years newly diagnosed with non-inflammatory disease may be eligible for enrollment in the cohort. For each of these subjects, a diagnosis of non-inflammatory disease may be made based on standard histologic, radiographic, and other features. A set of a first number of patients with non-inflammatory disease and a second number of non-disease controls from the cohort may be included in this analysis. Written informed consent may be provided by all parents or caregivers, and written assent may be obtained from children as appropriate.

Genotyping of the pediatric cohort may be performed at laboratories using ImmunoChip. A similar QC procedure may be performed, including assessment of individual and genotype missingness, allele frequencies, deviations from Hardy-Weinberg Equilibrium, gender check, and relatedness.

The performance of the DL algorithm and LDpred approach may be examined in the pediatric cohort, an independent cohort comprising newly-diagnosed pediatric non-inflammatory disease patients and non-disease controls genotyped using ImmunoChip. With a large percentage of the variants in the training model successfully genotyped, AUC values may be determined in DL-known, in DL-others, and in DL-comb, all of which may be significantly higher than that of LDpred. Although the DL model may be trained in a mostly adult cohort, results in the pediatric cohort may confirm its robustness in an independent and heterogenous test cohort.

Part of the reason underlying the superior performance of deep learning algorithm in genomic prediction may be that it may incorporate complex non-linear relationships in the model, which is largely ignored in most main-stream genomic prediction approaches. This may be particularly clear in DL-known algorithm in which only known variants (and variants in LD with known variants) are included. A linear prediction model in the training set may be built with those variants using step-wise logistic regression, and an AUC may be observed in the test cohort, which is lower than that of DL-known. This result may indicate that non-linear genetics effects may contribute significantly to phenotypic variance, which may be consistent with observations that high-order interactions contribute significantly to complex traits in model organisms. Although with the nature of DL algorithms it may be challenging to detangle details of the non-linear relationships, the potential high-order combination effects may be explored within known variants using LAMPlink, and a number of multiple SNP combinations (e.g., 3-SNP combinations) may be identified that are significantly associated with disease in both the non-inflammatory disease cohorts. Further, interesting cases of strong non-linear effects may be observed.

Interestingly, individuals carrying certain genetic variants may be identified as strongly associated with non-inflammatory disease, as indicated by OR. In non-inflammatory disease cases, subjects having the genetic variants may have much higher estimated risk in DL-known compared to LDPred and traditional PRS. As used herein, “eQTL” refers to expression quantitative trait loci, which shows an association of genetic variants with expression levels of mRNA.

As another example, individuals carrying certain genetic variants may have a high risk of non-inflammatory disease compared to non-carriers, as indicated by OR. LAMPlink analysis also may indicate strong deviation from linear additive model for the genetic variant. A high percentage of the homozygous risk (I/I) individuals in the non-inflammatory disease cohort may be non-inflammatory disease cases, corresponding to an OR of greater than 1.0 compared to wild type. Consistently, a large percentage of homozygous risk individuals may be non-inflammatory disease cases in the non-inflammatory disease cohort, as indicated by OR.

As used herein, “OR” refers to an odds ratio, which quantifies the strength of an association between two events. When OR is greater than 1, the two events are positively correlated; when the OR that is less than 1, the two events are negatively correlated. “P” as used herein refers to p value, which is the statistical significance of an association. A lower p value indicates a stronger statistical significance of the association than a p value that is higher. Carrier status refers to the number of risk variants (0, 1, 2, or 3) carried by subjects of each cohort. Results may be expressed along with a confidence interval (CI), such as a 95% confidence interval.

### Example 3: Association of Predicted Risk Scores with Clinical Characteristics of Non-Inflammatory Disease

Further, the association of predicted risk scores with clinical characteristics of non-inflammatory disease may be examined. Overall, the risk score calculated using DL-comb may have the strongest association with disease severity, disease location, and need for surgery. For example, in disease location analysis, OR values of much greater than 1.0 may be observed for DL-comb score, for DL-known, and for DL-others, and all three OR values may be greater than that for LDpred.

A strong association of the DL score with clinical phenotypes of non-inflammatory disease may be observed. Interestingly, although the prediction performance of DL-others may be worse than DL-known, the association of risk score from DL-others with disease severity may be comparable or sometimes, stronger. Although some unknown genetic variants may contribute weakly to non-inflammatory disease pathogenesis, they may have a relatively stronger effect on disease severity. This may indicate shared but different underlying genetic mechanisms of disease pathogenesis and progression.

Also contemplated herein, are DL models that account for demographic, behavioral, as well as other clinical relevant factors (e.g., duration of disease and treatment information) to further tailor prediction for clinical behavior and prognosis of non-inflammatory disease. Predictions for clinical behavior and prognosis of non-inflammatory disease may be leveraged to develop highly personalized treatment strategy and intervention, transforming non-inflammatory disease clinical practice.

Current analysis may be based on ImmunoChip, a platform enriched with immune-disease related variants. Further studies exploring performance of the deep learning algorithm in genome-wide data may be performed. Environmental factors may be incorporated into the prediction models to further improve the prediction performance, such as smoking information for the cases and controls in the training and test sets. Further, further development of the proposed approach toward improved differential diagnosis of non-inflammatory disease may improve the prediction model, in particular with non-inflammatory diseases with similar clinical presentations.

In this study, a prediction model of non-inflammatory disease risk may be constructed using Deep Learning algorithms using genetic data from non-inflammatory disease cohorts. A deep learning (DL)-based algorithm may be applied to predict disease status of non-inflammatory disease, and its performance may be compared to the popular LDPred approach. A training model may be built using a convolutional neural network (CNN) with hundreds or thousands of individuals in the non-inflammatory disease ImmunoChip cohort. The performance of this model may be validated in independent cohorts and in a pediatric inception cohort. In an independent test cohort of hundreds or thousands of individuals, a non-trivial improvement in prediction performance of DL may be observed, with an Area Under the Curve (AUC) value that is greater than that using the LDPred approach. This may be among the best performance of risk prediction of complex human diseases using genetic data, and a significant improvement on the popular LDPred approach. The improvement in prediction accuracy from the DL approach may lead to greatly enriched non-inflammatory disease cases in the extreme of the DL score. This finding may indicate that ostensibly “healthy” individuals in the top extremes of DL score may benefit from screening for evidence of non-inflammatory disease, and may provide an opportunity to study high-risk patients with preventive strategies, as may be shown in a number of non-inflammatory diseases. By producing such a high Odds Ratio (OR), DL-based approaches may enable cost-effective genetic screening (e.g., to a general population or a high-risk population such as individuals with family history and/or symptoms of non-inflammatory disease) in the extremes of DL prediction. The DL-based prediction approaches disclosed herein may be expanded to other complex diseases, and may promote early detection and prevention of complex human diseases, such as non-inflammatory disease.

Using only the known variants, the DL based algorithm (DL-known) may achieve a high AUC. Further analyses may indicate that in the known variants, the improved performance of the DL score is likely due to its ability to incorporate complex non-linear relationships of associated disease variants with disease phenotype. Moreover, after excluding known variants (and variants in LD with known), a high AUC may be observed for DL algorithm (DL-other). Variance importance metrics of the DL-other algorithm may identify a number of novel non-inflammatory disease variants that reached genome-wide significance in a meta-analysis incorporating thousands of individuals. DL predicted risk score may also be strongly associated with disease clinical phenotypes of non-inflammatory disease including disease location, severity and need for surgery. The corresponding prediction algorithm may be incorporated as a package (GeneticDL) in R.

The superior performance of Deep Learning algorithm in genomic prediction may be partly due to the fact that it may incorporate complex non-linear causal effects, which may be largely ignored in most mainstream genomic prediction approaches. This may be particularly clear with the dominant performance of the DL-known algorithm in which only known variants (and variants in LD with known variants) are included. Although it may be challenging to detangle details of the non-linear relationships given the nature of DL algorithms, the potential high-order combination effects within known variants may be examined using LAMPlink. Interesting deviations from linear additive model may be identified, including the combination effects of certain genetic variants. Functional work may demonstrate that certain genetic variants may act in tandem or synergistically to induce a non-inflammatory disease phenotype, indicating a potential biological mechanism for the observed non-linear effects. All the individuals that may be affected by the potential deviation from linear effects may have higher predicted risk in DL in comparison to LDPred, which may indicate that the performance of DL prediction may be partially explained by its ability to capture non-linear causal effects. This further may demonstrate that non-linear genetics effects may contribute significantly to phenotypic variance of complex diseases such as non-inflammatory disease, consistent with findings that higher-order interactions contribute significantly to complex traits in model organisms.

A Dense Neural Network analysis using the non-inflammatory disease ImmunoChip dataset may be performed. One factor that may affect the performance of the Machine Learning approaches is the data QC procedures. Stringent QC procedures may be applied to the training dataset, resulting in a relatively smaller number of SNPs considered. In spite of that, better overall performance may be achieved using the Deep Learning Model. This difference may be attributed to the following details in the design and algorithms which may enable significant technical improvements to the Deep Learning models. First, although studies may generally use Neural Network-based algorithms in prediction, the use of the CNN algorithm may be particularly advantageous, because the CNN automatically includes two data pre-processing layers (Convolutional Layer and Pooling Layer) that perform much of the computational heavy lifting before the fully-connected layers. As a comparison, a manual SNP preselection step based on single-SNP level statistics may be performed in typical Neural Network-based algorithms to reduce the dimension of data, which may potentially lead to loss of information. Second, intensive tuning of the hyperparameters may be performed in the Deep Learning Model, rather than using arbitrarily selected numbers of neurons and/or layers. Tuning of the parameters in Deep Learning Models may have important impact on performance of the models. Third, Deep Learning models may be constructed separately on known SNPs (as well as SNPs in LD, DL-known) and the rest of ImmunoChip (DL-others), rather than fitting all the pre-selected SNPs into the machine learning models; as a result, a superlearner (DL-comb) may be constructed by combining the two resulting models.

The analysis results may demonstrate consistently observed patterns of deviation from simple additive models in both training and the independent test cohort, which may indicate the advantages of incorporating non-linear effects in Deep Learning models for prediction of complex diseases such as non-inflammatory disease.

Deep Learning-based algorithms may be effectively utilized to predict non-inflammatory disease risk using genetic data. Results may demonstrate that this algorithm may significantly increase the prediction accuracy, and that the predicted disease risk may be associated with disease clinical characteristics. With decreasing costs and likely increased availability of next-generation sequencing data that are coupled to electronic health records, results such as these may highlight opportunities for the clinical utility of large-scale genomic data for common non-inflammatory diseases. Further, ethical frameworks and mechanisms to incorporate advances in genomic medicine for complex diseases into clinical practice may be developed.

### Example 4: Using DeepLearning and Genetic BigData to Predict Non-Inflammatory Disease

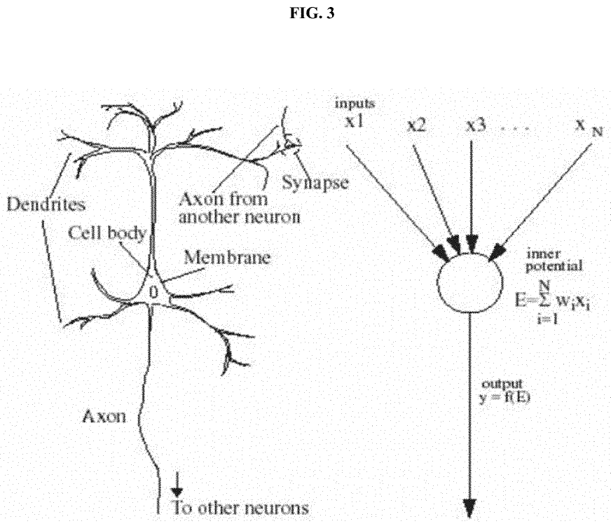

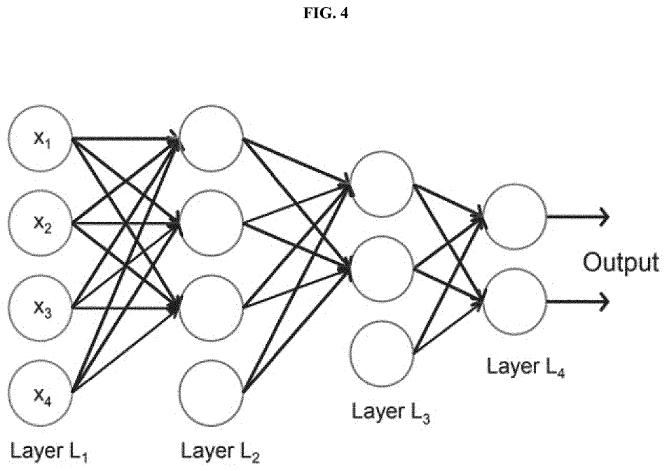

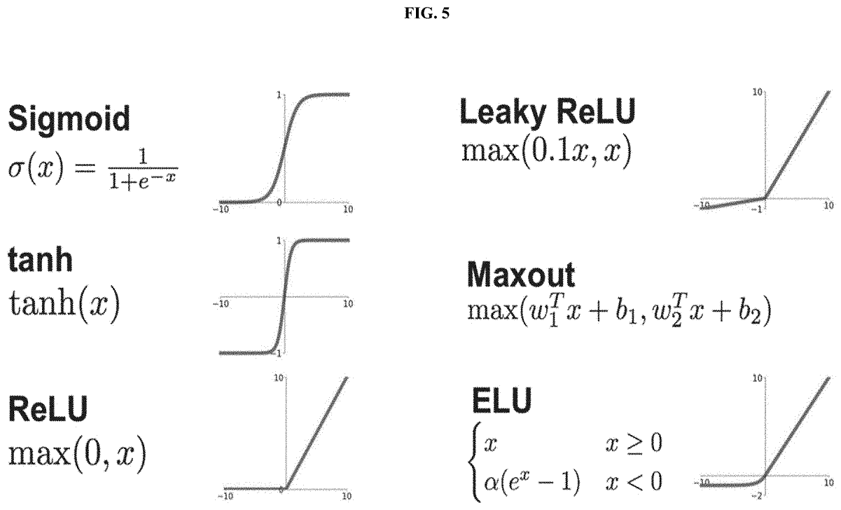

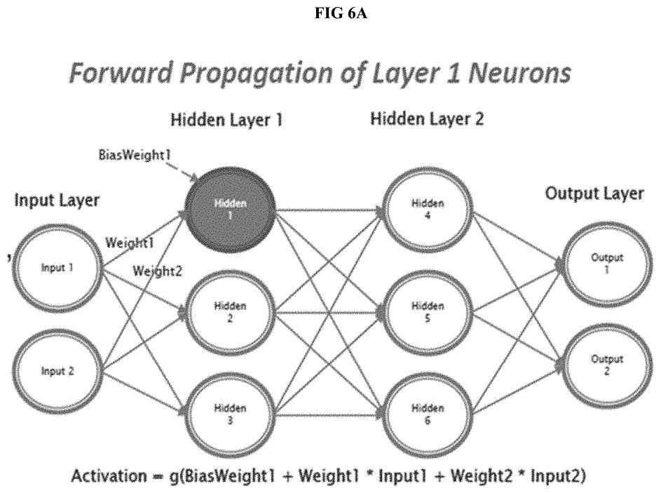

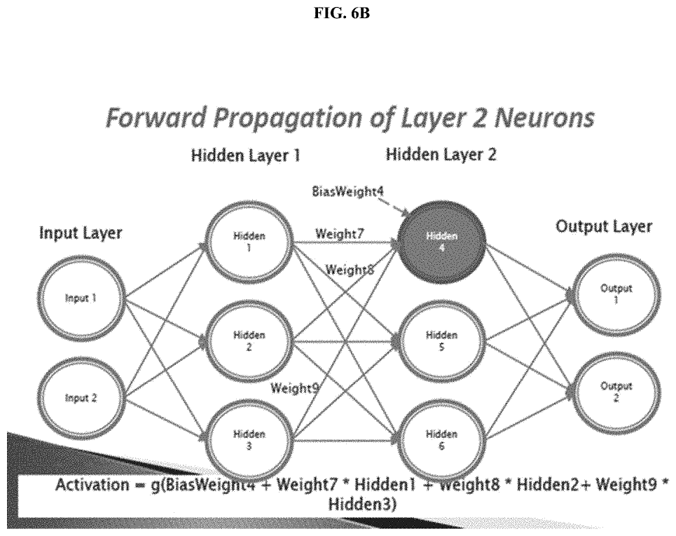

FIG. 3 shows a non-limiting example of a DeepLearning algorithm based on neural networking (similar to a brain's neurons), using the methods and systems disclosed herein. FIG. 4 shows a non-limiting example of DeepLearning algorithms using deep layers of neurons having an input layer, an output layer, and multiple intermediate layers between the input and output layers, using the methods and systems disclosed herein. FIG. 5 shows a non-limiting example of activation functions (e.g., fixed mathematical operations) that may be used in DeepLearning algorithms, such as sigmoid, tanh, ReLU, leaky ReLU, maxout, and ELU, using the methods and systems disclosed herein. FIGS. 6A-6B show non-limiting examples of forward propagation and backpropagation of a DeepLearning algorithm, using the methods and systems disclosed herein. During the forward propagation stage (FIG. 6A), features are input into the network and fed through the subsequent layers to produce the output activations. However, the error of the network can be calculated only at output units but not in the middle/hidden layers. In order to update the weights to optimal, the network errors are propagated backwards through its layers (FIG. 6B).

In some embodiments, LAMPlink is applied to compare disease risk in carriers of combinations of variants vs. the rest of the population. For example, a number of 3-variant combinations may be identified using LAMPlink.

Some combinations of genetic variants (e.g., SNPs) may indicate non-linear effects. Data results may be obtained that indicate deviations from a linear additive model.

In summary, an improved prediction model of non-inflammatory disease status may be developed based on genetic data, using DeepLearning approaches. There may be a monogenic level of risk in extreme of DL score. Also, DL score may have a strong association with clinical characteristics. DeepLearning approaches may demonstrate superior performance to LDpred, likely due to capturing the complex non-linear effects of causal variants, indicating there may be much more than linear additive effects in complex diseases.

### Example 5: Convolutional Neural Network Models

Using systems and methods of the present disclosure, convolutional neural network (CNN) prediction models may be constructed. The CNN models may comprise alternate layers of convolution and pooling followed by a fully connected layers (output) at the end. Batch normalization and dropout may also be incorporated to optimize the performance of the CNN.

The convolutional layer may comprise a set of convolutional kernels where each neuron acts as a kernel. The convolutional kernel may work by dividing the data into small slices which helps in extracting feature motifs. The kernel may convolve using a specific set of weights by multiplying its elements with the corresponding elements of the receptive field. The convolution operation may be expressed by the following expression:

fₗ^{k}(p,q)=Σ_{c}Σ_{x,y}i_{c}(x,y)eₗ^{k}(u,v)

Here, i_{c}(x, y) may be an element of the input data i_{c}, which may be element wise multiplied by eₗ^{k}(u, v) index of the kth convolutional kernel k^{l} of the lth layer. The output feature-map of the kth convolutional operation may be expressed by the following expression:

Fₗ^{k}=[fₗ^{k}(1,1), . . . ,fₗ^{k}(p,q), . . . ,fₗ^{k}(P,Q)]

The CNN may comprise a pooling layer to perform pooling or down-sampling.

Feature motifs, which may result as an output of convolution operation, may occur at different locations in the data. Once features are extracted, its exact location may become less important as long as its approximate position relative to others is preserved. Pooling or down-sampling may be a local operation that sums up similar information in the neighborhood or proximity of the receptive field and outputs the dominant response within this local region. This operation may be expressed by the following expression:

Zₗ^{k}=φp(Fₗ^{k})

Here Zₗ^{k} may represent the pooled feature-map of the lth layer for the kth input feature-map Fₗ^{k}, whereas φₚ may define the type of pooling operation. The use of the pooling operation may help to extract a combination of features, which may be invariant to translational shifts and small distortions. A reduction in the size of feature-map to invariant feature set may not only regulate the complexity of the network, but also help in increasing the generalization by reducing overfitting. Max, Average, and/or Overlapping may be used as the pooling formulation in model optimization.

The CNN may comprise an activation function, which serves as a decision function and helps in learning of intricate patterns. The selection of an appropriate activation function may accelerate the learning process. The activation function may be defined using the following expression:

Tₗ^{k}=φₐ(Fₗ^{k})

Here, Fₗ^{k} may be an output of a convolution, which may be assigned to activation function φₐ that adds non-linearity and returns a transformed output Tₗ^{k} for the lth layer. Activation functions including sigmoid, tanh, maxout, and ReLU may be evaluated for selection when tuning or optimizing the neural network.

Batch normalization may be performed on the CNN to address the issues related to the internal covariance shift within feature-maps. The internal covariance shift may be a change in the distribution of hidden units' values, which may slow down the convergence (by forcing learning rate to small value) and require careful initialization of parameters. Batch normalization for a transformed feature-map Fₗ^{k} may be calculated using the following expression:

$${N}_{l}^{k} = \frac{{F}_{l}^{k} - {μ}_{B}}{\sqrt{{σ}_{B}^{2} + ε}}$$

Here, Nₗ^{k} may represent normalized feature-map, Fₗ^{k} may be the input feature-map, and μ_{B} and σ_{B}² may represent mean and variance of a feature-map for a mini batch, respectively. In order to avoid division by zero, ε may be added for numerical stability. Batch normalization may unify the distribution of feature-map values by setting them to zero mean and unit variance. Further, it may smoothen the flow of gradient and act as a regulating factor, which may help in improving the generalization of the neural network.

Dropout may be performed on the CNN to introduce regularization within the neural network, which may improve generalization by randomly skipping some units or connections with a certain probability. This random dropping of some connections or units may produce several thinned neural network architectures, and finally, one representative neural network is selected with small weights. This selected neural network architecture may be then considered as an approximation of all of the proposed neural networks. In the CNN model, the dropout ratio may be optimized using grid search as a hyperparameter.

The CNN may comprise fully connected layers (e.g., output layers), which may be used at the end of the neural network for classification and/or prediction. Unlike pooling and convolution, it may be a global operation. It may take input from feature extraction stages and globally analyze the output of all the preceding layers.

### Example 6: R Software Program Configured to Perform the DeepLearning Algorithm

Using systems and methods of the present disclosure, an R software program may be configured to perform the DeepLearning algorithm. The R software program, which may be stored on a non-transitory computer-readable medium, may comprise machine-executable code that, upon execution by one or more computer processors, implements numerous operations of an example of a DeepLearning algorithm, including constructing a DeepLearning model, calculating the performance of the DeepLearning algorithm, performing phenotype analysis using a set of predictor SNPs (e.g., known SNPs that are associated with an non-inflammatory disease), training the DeepLearning model by performing prediction of the non-inflammatory disease using a training dataset, performing prediction of the non-inflammatory disease using a test dataset, performing cross-validation, and constructing a combined DeepLearning model out of a plurality of separate DeepLearning models.

An example of code for an R software program configured to perform the DeepLearning algorithm is provided below.

```
  ########## Begin R code software program
  rm(list=ls(all=T))
  work.dir<- getwd( )
  setwd(work.dir)
  Interm.dir = paste(work.dir,“Interm/”,sep=“”)
  dir.create(Interm.dir)
  res.dir = paste(work.dir,“results/”,sep=“”)
  dir.create(res.dir)
  dir.create(paste(res.dir,“/cohort-name/best/”,sep=“”)
  library(h2o)
  library(e1071)
  ##### Re-calculate the performance of the DL algorithm in family ctrls
  h2o.shutdown( )
  ########## Analysis starts
  ###### First, prediction using known SNPs
  h2o.init(nthreads=−1,max_mem_size=“1000G”)
  name.known.tr2.g=paste(Interm.dir,“g_known_tr2.csv”,sep=“”)
  name.known.va.g=paste(Interm.dir,“g_known_va.csv”,sep=“”)
  name.known.tr.g=paste(Interm.dir,“g_known_tr.csv”,sep=“”)
  name.known.te.g=paste(Interm.dir,“g_known_te.csv”,sep=“”)
  dat.known.tr=h2o.importFile(name.known.tr.g,“dat.known.tr”)
  dat.known.tr2=h2o.importFile(name.known.tr2.g,“dat.known.tr2”)
  dat.known.te=h2o.importFile(name.known.te.g,“dat.known.te”)
  dat.known.va=h2o.importFile(name.known.va.g,“dat.known.va”)
  dat.known.tr$PHENOTYPE=as.factor(dat.known.tr$PHENOTYPE)
  dat.known.tr2$PHENOTYPE=as.factor(dat.known.tr2$PHENOTYPE)
  dat.known.va$PHENOTYPE=as.factor(dat.known.va$PHENOTYPE)
  dat.known.te$PHENOTYPE=as.factor(dat.known.teSPHENOTYPE)
  snps.known=read.table(paste(Interm.dir,“known_snps_list.txt”,sep=“”))
  ##### Analysis using DeepLearning
  response=“PHENOTYPE”
  predictors=snps.known[,1]
  dat.known.split=h2o.splitFrame(dat.known.tr)
  dat.known.tr2=dat.known.split[1]]
  dat.known.va=dat.known.split[2]]
  ########## Run the DeepLearning model (takes several days' runtime)
  m_165_2_cv_known=h2o.deeplearning(model_id=“m_165_2_cv_known”
  training_frame=dat.known.tr,
      #validation_frame=dat.known.te,
      x=predictors,y=response,
      hidden=c( 165,165),
      activation=“MaxoutWithDropout”,
      #activation=“Rectifier”,
      #activation=“ Maxout”,
      input_dropout_ratio=0.1,
      epochs=10,
      overwrite_with_best_model=T,
      nfold=10,
      fold_assignment=“AUTO”,
      l1=1.1E−4,
      l2=6.0E−5,
      #loss = “CrossEntropy”,
      keep_cross_validation_predictions = TRUE,
      #missing_values_handling = “Meanimputation”,
      score_validation_samples=10000,
      stopping_round=2,
      stopping_metric=“AUC”,
      stopping_tolerance=0.0001,
      seed=1
  )
  summary(m_165_2_cv_known)
  name.known.te.info=paste(Interm.dir,“info_known_te.csv”,sep=“”)
  info.known.te=read.table(name.known.te.info,sep=“,”)
  name.known.tr.info=paste(Interm.dir,“info_known_tr.csv”,sep=“”)
  info.known.tr=read.table(name.known.tr.info,sep=“,”)
  ########## Perform prediction in training dataset
  cros.pred=h2o.cross_validation_predictions(m_165_2_cv_known)
  pred={ }
  for(i in 1:5){
    pred.i=as.vector(((cros.pred[[i]])$p1)$p1)
    pred=cbind(pred,pred.i)
  }
  pred.dl.known=rowMeans(pred)*5
  #pred.dl.known=as.vector((pred.dl.known$p1)$p1)
  info.known.tr2=cbind(info.known.tr,pred.dl.known)
  colnames(info.known.tr2)[7]=“pred_dl_known”
  ########## Perform prediction in test dataset
  pred.v.known.dl=h2o.predict(m_165_2_cv_known,newdata=dat.known.te)
  name.known.te.info=paste(Interm.dir,“info_known_te.csv”,sep=“”)
  info.known.te=read.table(name.known.te.info,sep=“,”)
  pred.dl.known=as.vector((pred.v.known.dl$p1)$p1)
  info.known.te=cbind(info.known.te,pred.dl.known)
  colnames(info.known.te)[7]=“pred_dl_known”
  ##### Save the constructed DeepLearning model
  h2o.saveModel(m_165_2_cv_known,path=paste(res.dir,“/ cohort-
name/best/”,sep=“”),force=T)
  #m_l 65_2_cv_known=h2o.loadModel(path=paste(res. dir,“/cohort-
name/best/m_l 65_2_cv_known”,sep=“”))
  ########## Obtain the best performing DeepLearning model
  #auc.i=h2o.auc(m_160_2_cv_known)
  ############################################
  ########## Perform prediction using the rest of iChip
  ############################################
  name.other.tr.g=paste(Interm.dir,“g_others_tr.csv”,sep=“”)
  name.other.te.g=paste(Interm.dir,“g_others_te.csv”,sep=“”)
  dat.other.tr=h2o.importFile(name.other.tr.g,“dat.other.tr”)
  dat.other.te=h2o.importFile(name.other.te.g,“dat.other.te”)
  dat.other.tr$PHENOTYPE=as.factor(dat.other.tr$PHENOTYPE)
  snps.other=read.table(paste(Interm.dir,“others_snps_list.txt”,sep=“”))
  ########## Perform analysis using DeepLearning
  response=“PHENOTYPE”
  predictors=snps.other[,1]
  dat.other.split=h2o.splitFrame(dat.other.tr)
  dat.other.tr2=dat.other.split[[1]]
  dat.other.va=dat.other.split[[2]]
  ##### Note: this takes weeks of runtime, even on a powerful workstation
  m_325_3_cv_others=h2o.deeplearning(model_id=“m_325_3_cv_other”,
      training_frame=dat.other.tr,
      #validation_frame=dat.te,
      x=predictors,y=response,
      hidden=c(325,325,325),
      #activation=“Tanh”,
      activation=“RectifierWithDropout”,
      input_dropout_ratio=.1,
      epochs=10,
      overwrite_with_best_model=T,
      nfold=5,
      fold_assignment=“AUTO”,
      l1=6.0E−5,
      l2=1.0E−4,
      keep_cross_validation_predictions = TRUE,
      score_validation_samples=10000,
      stopping_round=2,
      stopping_metric=“AUC”,
      stopping_tolerance=0.001
  )
  ##### Obtain the predictions
  name.others.te.info=paste(Interm.dir,“info_others_te.csv”,sep=“”)
  info.others.te=read.table(name.others.te.info,sep=“,”)
  name.others.tr.info=paste(Interm.dir,“info_others_tr.csv”,sep=“”)
  info.others.tr=read.table(name.others.tr.info,sep=“,”)
  ##### In training set
  cros.pred=h2o.cross_validation_predictions(m_325_3_cv_others)
  pred={ }
  for(i in 1:5){
    pred.i=as.vector(((cros.pred[[i]])$p1)$p1)
    pred=cbind(pred,pred.i)
  pred.dl.others=rowMeans(pred)*5
  #pred.dl.others=as.vector((pred.dl.others$p1)$p1)
  info.others.tr2=cbind(info.others.tr,pred.dl.others)
  colnames(info.others.tr2)[7]=“pred_dl_others”
  ##### In test set
  pred.v.others.dl=h2o.predict(m_325_3_cv_others,newdata=dat.other.te)
  pred.dl.others=as.vector((pred.v.others.dl$p1)$p1)
  info.others.te2=cbind(info.others.te,pred.dl.others)
  colnames(info.others.te2)[7]=“pred_dl_others”
  ########## DeepLearning combined
  info_comb_tr =
merge(info.known.tr[,c(“FID”,“PHENOTYPE”,“pred_dl_known”)],info.others.tr[,c(“FID”,“p
red_dl_others”)])
  info_comb_tr$PHENOTYPE = as.factor(info_comb_tr$PHENOTYPE)
  model_comb = svm(PHENOTYPE~pred_dl_others+pred_dl_known,data =
info_comb_tr,kemal = “radial”,epsilon = 0.05, tolerance = 5E−4)
  info_comb_te =
merge(info.known.te[,c(“FID”,“PHENOTYPE”,“pred_dl_known”)],info.others.te[,c(“FID”,“p
red_dl_others”)])
  #info_comb_te$PHENOTYPE = as.factor(info_comb_te$PHENOTYPE)
  predict(model_comb,newdata = info_comb_te)
```

### Example 7: Using DeepLearning and Genetic BigData to Construct a Disease Prediction Model for Fibrosis in Primary Sclerosing Cholangitis (PSC)

Using systems and methods of the present disclosure, a Deep Learning (DL) model is built, validated and tested to predict fibrosis in subjects suffering from primary sclerosing cholangitis (PSC) using genetic data. The performance of the DL model in this example is compared to the performance of LDpred. The DL model in this example and according to various embodiments described herein will yield more accurate predictions as compared to LDpred, underscoring the clinical utility of the DL model in clinical practice to inform decision-making (e.g., diagnosis, prognosis, selection of therapeutic intervention, disease and/or therapeutic regimen monitoring, and the like).

Methods: DL is utilized to build a disease prediction model with cases and controls selected from the UK-PSC Consortium, the International IBD Genetics Consortium, the International PSC Study Group, and the Cedars-Sinai MIRIAD cohort. 4,796 PSC cases and 19,955 non-PSC controls are selected from the UK-PSC Consortium, the International IBD Genetics Consortium, and the International PSC Study Group, as the training dataset. This model will be further validated in 312 PSC cases and 6,336 non-PSC controls from the Cedars-Sinai MIRIAD cohort, that are independent from the training set. Both training and validation cohorts are genotyped using ImmunoChip. A set of 7.9 million variants that are successfully measured and passed the stringent QC are included as predictors. A convolutional neural network (CNN) algorithm is used to construct a DL model, and cross-validation is performed as part of the DL model construction. Further, the association of the DL prediction score is examined with clinical phenotypes.

Performance of the DL model is compared to the LDpred algorithm (e.g., as described by Amit V. Khera et. al, Nature Genetics, 2018; which is incorporated herein by reference in its entirety). A non-trivial improvement in prediction performance of DL is observed with an Area Under the Curve (AUC) of about 0.875, as compared to about 0.7 using LDPred. The predicted risk from DL will lead to greatly enriched cases in the extreme of the DL score, with an OR of 30 with a P=1E-20 in the validation cohort. Utilizing only a set of about 2,000 known disease-susceptibility variants (and variants in LD with known), the DL based algorithm (DL-known) will achieve an AUC of about 0.875. Further analyses will indicate that the improved performance of the DL-known score is likely through its ability to incorporate non-linear causal effects. Moreover, after excluding known variants, an AUC of about 0.75 will be observed with the DL algorithm (DL-other). Variance importance metrics of the DL-other algorithm will identify novel variants that will achieve a genome-wide significance in a meta-analysis incorporating a large set of over 100,000 individuals. Via DL analysis, the expected predicted risk score will be also strongly associated with PSC clinical phenotypes including disease location, severity, and need for therapeutic interventions. Therefore, utilizing this genetic algorithm, individuals with disease risk for PSC will be identified, a capability that provides progress towards early diagnosis and identifying subjects for studying preventative strategies.

Subjects will be enrolled as follows. A training cohort will be obtained, comprising individuals from the UK-PSC Consortium, the International IBD Genetics Consortium, and the International PSC Study Group. Subject recruitment will include recruiting a set of 4,796 cases and 19,955 controls. For each of these subjects, a diagnosis of PSC will be made based on accepted clinical evaluation (e.g., radiological, endoscopic, and histopathological evaluation). All included cases will fulfill clinical criteria for PSC and provided written consent. The entire cohort (the UK-PSC Consortium, the International IBD Genetics Consortium, and the International PSC Study Group), after excluding any overlap with a test cohort, will be used as a training dataset.

An independent cohort from Cedars-Sinai Medical Center (CSMC) MIRIAD will be used as a test cohort to generate a test dataset. Validation will be performed in this test cohort of 312 PSC cases and 6,336 non-PSC controls. The patient recruitment for the Cedars cohort will include PSC cases and non-PSC control cases with genotype data (after QC). For each of these subjects, a diagnosis of PSC will be made based on standard clinical features (e.g., endoscopic, histologic, and radiographic features). The study protocol and data collection, including DNA preparation and genotyping, will be approved by the CSMC Institutional Review Board. Written informed consents will be obtained from all study participants.

Genotyping and genotype quality control (QC) will be performed as follows. All cohorts will be genotyped using Illumina ImmunoChip™ platform. Further, QC in the cohorts will be performed. In brief, the ImmunoChip™ samples will be genotyped in 36 batches, and genotype calling will be performed separately for each batch. Stringent QC will be performed, removing the following: SNPs with a call rate lower than 98% across all genotyping batches or 90% in one of the genotyping batches, SNPs that do not appear in the 1000 Genomes Project Phase I, SNPs that failed Hardy-Weinberg Equilibrium (P<10⁻⁵ across all samples or within each genotyping batch), and monomorphic SNPs. Individuals will be assigned to different populations based on principal components, and those not in the European Ancestry cluster, and those with low call rate (less than 98%), outlying heterozygosity rate (P<0.01) or cryptic relatedness (identity by decent >0.4) will be removed.

Genotyping of the Cedars cohort will be performed at CSMC using an Illumina ImmunoChip™ array. Individual and genotype missingness, allele frequencies, and deviations from Hardy-Weinberg Equilibrium will be calculated using the PLINK software package (pngu.mgh.harvard.edu/˜purcell/plink). Individual-level QC thresholds will be used, including a genotyping call rate of greater than 95% and an inbreeding coefficient of less than 0.05. Ethnicity outliers that are identified using Admixture software will also be removed. SNPs with a call rate of less than 0.95, minor allele frequency (MAF) of less than 0.01, and strong deviation from Hardy-Weinberg equilibrium (P<10⁻⁷) will also be removed.

A set of 7.9 million SNPs available post-QC in the UK-PSC Consortium, the International IBD Genetics Consortium, and the International PSC Study Group cohorts, will be selected for further analyses. Of these, about 2,000 are known PSC variants or in LD with known PSC variants with r2>0.2 in 1000 Genome Project Phase3 data.

The Deep Learning prediction models will be constructed as follows. A multi-layer feedforward artificial neural network, also known as convolutional neural network (CNN), will be utilized to build the prediction model. The CNN model will be constructed separately with a) the 2,000 variants that are either known or in LD (r2>0.2) with known PSC variants (DL-known), and b) the remaining variants not in LD with known variants (DL-others). The CNN model will be optimized in the software H2O using stochastic gradient descent with both L₁ and L₂ regularization. A grid search will be performed to determine the best parameter settings separately for DL-known and DL-others, including numbers of hidden layers, number of neurons in each layer, activation functions of the layers, dropout ratio, and parameters for L₁ and L₂ regularization. In the trained models, the variable relative importance will be calculated using Gedeon's approach, based on the weights connecting the input features to the first two hidden layers. A 5-fold cross-validation will be applied to the control for model overfitting. Further, an ensemble model (DL-comb) based on Support Vector Machine (SVM) will be built to combine DL-known and DL-others with 5-fold cross-validation. After building up different Deep Learning models in the training dataset, models are fitted using the test datasets. The final prediction model will be incorporated into a PSC risk prediction tool.

Prediction performance of the deep learning algorithm will be compared to the LDPred approach as follows.

LDpred analysis will be performed using the default parameters, based on the public available summary statistics. The LDPred23 Python package will be used for these analyses. LDPred analysis will be performed across different p-value thresholds (1.0E-6, 3.0E-6, 1.0E-5, 3.0E-5, 1.0E-4, 3.0E-4, 0.001, 0.003, 0.01, 0.05, 0.10, and 0.25), and the p-value threshold with best AUC will be selected.

Prediction performance will be evaluated as follows. Receiver Operating Characteristic (ROC) curves will be generated for different prediction models in the test dataset. Further, Area Under Curve (AUC) will be calculated for each of the ROC curves, and compared using the R package pROC31. Further, the performance of difference approaches will be evaluated in enrichment of PSC cases in the extreme of PSC risk prediction. All comparisons will be performed in the R software package.

High-order combination analysis will be performed as follows. To investigate the effects of non-linear effects in known variants (and variants associated with known), the combined effects of variants used in DL-known analysis will be examined using LAMPlink software. Combinations of both dominant and recessive models are performed, and LD filtering will be performed with r2 cutoff of 0.2 to exclude potential contamination from SNPs in strong LD.

Association of single variants with PSC and meta-analysis will be performed as follows. Association of SNPs within the top 500 of variable importance with PSC will be examined in the UK-PSC Consortium, the International IBD Genetics Consortium, the International PSC Study Group, and the Cedars-Sinai MIRIAD cohorts separately, using logistic regression with adjustment for principal components from population stratification analysis. A meta-analysis will be performed to combine the summary statistics in both cohorts, after excluding overlapping samples.

Association of predicted risk with PSC clinical phenotypes will be performed as follows. Association of prediction score from different algorithms with clinical characteristics will be evaluated in the generalized linear model framework, with Principal Components from population stratification analysis included as covariates.

Results

After performing an intensive grid search to tune or optimize the hyperparameters, a CNN model with three hidden layers (154 neurons in each layer, with L₁ penalty of 5.0E-5 and L₃ penalty of 1.0E-4) will be constructed for DL-known. For DL-others, a model with two hidden layers (326 neurons in each layer) with L₁ penalty of 6.0E-5 and L₂ penalty of 1.6E-4 will be constructed. A SVM model combining DL-known and DL-others will be then trained in the training cohort combining the DL-known and DL-others models.

A significant improvement in prediction performance will be observed using the deep learning algorithm compared to LDpred. In the test set, the Area Under the Curve (AUC) of the LDpred approach (with p-value cutoff of 0.01) will be about 0.7, while deep learning constructed using the known variants and variants in LD with known (DL-known) will exhibit an AUC of about 0.875, which will be significantly higher than that of LDpred. Deep learning with other variants (DL-others), where variants included in the DL-known analysis that will be excluded, will exhibit an AUC of about 0.75, which will be also higher than the LDpred prediction. Combining the DL-known and DL-other variants (DL-comb) will improve the overall AUC of prediction to about 0.875, which is among the best performance of risk prediction of complex human diseases using genetic data.

This improvement in prediction accuracy will lead to greatly enriched cases in both the extreme and even the not-so-extreme tails of the DL-comb scores. All deep-learning based approaches, whether based on the known variants, the remaining of the immunoChip, or the combined, will demonstrate better performance as compared to LDpred. For example, in the top 5% of the predicted risk, an Odds Ratio (OR) of about 10 will be observed for DL-others, an OR of about 20 to 25 will be observed for DL-known, and an OR of about 25 to 30 will be observed for DL-comb, compared to the rest of the 95% of the samples (as compared to about 6 for LDpred). As another example, in the top 10% of the predicted risk, observed Odds Ratios (OR) will be about 8 for DL-others, about 15-20 for DL-known, and about 20 for DL-comb, compared to about 4 for LDpred. Within the top 5% and 10% of the DL-comb score, about 90% will be PSC patients. As a comparison, in LDPred algorithm proportion of PSC patients will be about 80% and about 70%, respectively in the top 5% and 10%. The corresponding positive likelihood ratio (LR+) will be about 3 for DL-comb using top 5% and 10% cutoff, and about 2 for LDpred. And the corresponding negative likelihood ratio (LR−) will be about 0.1 to 0.2 for DL-comb, and about 0.3 to 0.5 for LDPred, respectively. These all indicate that the deep learning algorithms may greatly boost the practical potentials of genomic prediction.

Similar performance of the DL algorithm will be observed in additional cohorts, thereby confirming the DL model's robustness in independent and heterogenous test cohorts.

The variance importance metrics of the DL-other model in PSC prediction will be examined. The variable importance metrics from the DL algorithm indicates the relative importance or contribution of each variable and/or mutation to the overall model, which may be helpful in discovery of novel signals. For the top 500 variants, a meta-analysis will be performed incorporating the immunoChip data from the UK-PSC Consortium, the International IBD Genetics Consortium, the International PSC Study Group, and the Cedars-Sinai MIRIAD cohort. Of those 500 variants, it is expected that about 10 or more novel genome-wide signals will be identified with a meta-analysis p-value of about 1×10⁻⁹.

Conclusion: With this DL model, individuals with disease risk for a fibrotic disease, such as PSC, may be identified using only genetic data, making it a powerful tool for disease early diagnosis and intervention. The utility of the DL model is not limited to predicting complex disease risk for PSC. Without being bound by any particular theory, this DL model may be utilized with a wide range of genetic data input (e.g., genetic variants associated with other fibrotic diseases) to predict complex disease risk.

### Example 8: Using DeepLearning and Genetic BigData to Construct a Disease Prediction Model for Fibrosis in Scleroderma

Using systems and methods of the present disclosure, a Deep Learning (DL) model is built, validated and tested to predict fibrosis in subjects suffering from scleroderma using genetic data. The performance of the DL model in this example is compared to the performance of LDpred. The DL model in this example and according to various embodiments described herein will yield more accurate predictions as compared to LDpred, underscoring the clinical utility of the DL model in clinical practice to inform decision-making (e.g., diagnosis, prognosis, selection of therapeutic intervention, disease and/or therapeutic regimen monitoring, and the like).

Methods: DL is utilized to build a disease prediction model with cases and controls selected from the European Scleroderma Group, the Australia Scleroderma Group, and two US SSc cohorts. 8,231 scleroderma cases and 10,356 non-scleroderma controls are selected from the European Scleroderma Group and the Australia Scleroderma Group, as the training dataset. This model will be further validated in 1,615 scleroderma cases and 6,973 non-scleroderma controls from the two US SSc cohorts, that are independent from the training set. Both training and validation cohorts are genotyped using ImmunoChip. A set of 6.7 million variants that are successfully measured and passed the stringent QC are included as predictors after imputation. A convolutional neural network (CNN) algorithm is used to construct a DL model, and cross-validation is performed as part of the DL model construction. Further, the association of the DL prediction score is examined with clinical phenotypes.

Performance of the DL model is compared to the LDpred algorithm (e.g., as described by Amit V. Khera et. al, Nature Genetics, 2018; which is incorporated herein by reference in its entirety). A non-trivial improvement in prediction performance of DL is observed with an Area Under the Curve (AUC) of about 0.8, as compared to about 0.7 using LDPred. The predicted risk from DL will lead to greatly enriched cases in the extreme of the DL score, with an OR of 15 with a P=1E-15 in the validation cohort. Utilizing only a set of about 500 known disease-susceptibility variants (and variants in LD with known), the DL based algorithm (DL-known) will achieve an AUC of about 0.8. Further analyses will indicate that the improved performance of the DL-known score is likely through its ability to incorporate non-linear causal effects. Moreover, after excluding known variants, an AUC of about 0.7 will be observed with the DL algorithm (DL-other). Variance importance metrics of the DL-other algorithm will identify novel variants that will achieve a genome-wide significance in a meta-analysis incorporating a large set of over 100,000 individuals. Via DL analysis, the expected predicted risk score will be also strongly associated with scleroderma clinical phenotypes including disease location, severity, and need for therapeutic interventions. Therefore, utilizing this genetic algorithm, individuals with disease risk for scleroderma will be identified, a capability that provides progress towards early diagnosis and identifying subjects for studying preventative strategies.

Subjects will be enrolled as follows. A training cohort will be obtained, comprising individuals from the European Scleroderma Group and the Australia Scleroderma Group. Subject recruitment will include recruiting a set of 8,231 scleroderma cases and 10,356 non-scleroderma controls. For each of these subjects, a diagnosis of scleroderma will be made based on accepted clinical evaluation (e.g., radiological, endoscopic, and histopathological evaluation). All included cases will fulfill clinical criteria for scleroderma and provided written consent. The entire cohort (the European Scleroderma Group, the Australia Scleroderma Group, and two US SSc cohorts), after excluding any overlap with a test cohort, will be used as a training dataset.

An independent cohort from two US SSc cohorts will be used as a test cohort to generate a test dataset. Validation will be performed in this test cohort of 1,615 scleroderma cases and 6,973 non-scleroderma controls. The patient recruitment for the two US SSc cohorts will include scleroderma cases and non-scleroderma control cases with genotype data (after QC). For each of these subjects, a diagnosis of scleroderma will be made based on standard clinical features (e.g., endoscopic, histologic, and radiographic features). The study protocol and data collection, including DNA preparation and genotyping, will be approved by the Institutional Review Board. Written informed consents will be obtained from all study participants.

Genotyping and genotype quality control (QC) will be performed as follows. All cohorts will be genotyped using Illumina ImmunoChip™ platform. Further, QC in the cohorts will be performed. In brief, the ImmunoChip™ samples will be genotyped in 36 batches, and genotype calling will be performed separately for each batch. Stringent QC will be performed, removing the following: SNPs with a call rate lower than 98% across all genotyping batches or 90% in one of the genotyping batches, SNPs that do not appear in the 1000 Genomes Project Phase I, SNPs that failed Hardy-Weinberg Equilibrium (P<10⁻⁵ across all samples or within each genotyping batch), and monomorphic SNPs. Individuals will be assigned to different populations based on principal components, and those not in the European Ancestry cluster, and those with low call rate (less than 98%), outlying heterozygosity rate (P<0.01) or cryptic relatedness (identity by decent >0.4) will be removed.

Genotyping of the validation cohort will be performed using an Illumina ImmunoChip™ array. Individual and genotype missingness, allele frequencies, and deviations from Hardy-Weinberg Equilibrium will be calculated using the PLINK software package (pngu.mgh.harvard.edu/˜purcell/plink). Individual-level QC thresholds will be used, including a genotyping call rate of greater than 95% and an inbreeding coefficient of less than 0.05. Ethnicity outliers that are identified using Admixture software will also be removed. SNPs with a call rate of less than 0.95, minor allele frequency (MAF) of less than 0.01, and strong deviation from Hardy-Weinberg equilibrium (P<10⁻⁷) will also be removed.

A set of 6.7 million SNPs available post-QC in the European Scleroderma Group, the Australia Scleroderma Group, and two US SSc cohorts, will be selected for further analyses. Of these, about 500 are known scleroderma variants or in LD with known scleroderma variants with r2>0.2 in 1000 Genome Project Phase3 data.

The Deep Learning prediction models will be constructed as follows. A multi-layer feedforward artificial neural network, also known as convolutional neural network (CNN), will be utilized to build the prediction model. The CNN model will be constructed separately with a) the about 500 variants that are either known or in LD (r2>0.2) with known scleroderma variants (DL-known), and b) the remaining variants not in LD with known variants (DL-others). The CNN model will be optimized in the software H2O using stochastic gradient descent with both L₁ and L₂ regularization. A grid search will be performed to determine the best parameter settings separately for DL-known and DL-others, including numbers of hidden layers, number of neurons in each layer, activation functions of the layers, dropout ratio, and parameters for L₁ and L₂ regularization. In the trained models, the variable relative importance will be calculated using Gedeon's approach, based on the weights connecting the input features to the first two hidden layers. A 5-fold cross-validation will be applied to the control for model overfitting. Further, an ensemble model (DL-comb) based on Support Vector Machine (SVM) will be built to combine DL-known and DL-others with 5-fold cross-validation. After building up different Deep Learning models in the training dataset, models are fitted using the test datasets. The final prediction model will be incorporated into a scleroderma risk prediction tool.

Prediction performance of the deep learning algorithm will be compared to the LDPred approach as follows.

LDpred analysis will be performed using the default parameters, based on the public available summary statistics. The LDPred23 Python package will be used for these analyses. LDPred analysis will be performed across different p-value thresholds (1.0E-6, 3.0E-6, 1.0E-5, 3.0E-5, 1.0E-4, 3.0E-4, 0.001, 0.003, 0.01, 0.05, 0.10, and 0.25), and the p-value threshold with best AUC will be selected.

Prediction performance will be evaluated as follows. Receiver Operating Characteristic (ROC) curves will be generated for different prediction models in the test dataset. Further, Area Under Curve (AUC) will be calculated for each of the ROC curves, and compared using the R package pROC31. Further, the performance of difference approaches will be evaluated in enrichment of scleroderma cases in the extreme of scleroderma risk prediction. All comparisons will be performed in the R software package.

High-order combination analysis will be performed as follows. To investigate the effects of non-linear effects in known variants (and variants associated with known), the combined effects of variants used in DL-known analysis will be examined using LAMPlink software. Combinations of both dominant and recessive models are performed, and LD filtering will be performed with r2 cutoff of 0.2 to exclude potential contamination from SNPs in strong LD.

Association of single variants with scleroderma and meta-analysis will be performed as follows. Association of SNPs within the top 500 of variable importance with scleroderma will be examined in the European Scleroderma Group, the Australia Scleroderma Group, and two US SSc cohorts separately, using logistic regression with adjustment for principal components from population stratification analysis. A meta-analysis will be performed to combine the summary statistics in both cohorts, after excluding overlapping samples.

Association of predicted risk with scleroderma clinical phenotypes will be performed as follows. Association of prediction score from different algorithms with clinical characteristics will be evaluated in the generalized linear model framework, with Principal Components from population stratification analysis included as covariates.

Results

After performing an intensive grid search to tune or optimize the hyperparameters, a CNN model with three hidden layers (154 neurons in each layer, with L₁ penalty of 5.0E-5 and L₃ penalty of 1.0E-4) will be constructed for DL-known. For DL-others, a model with two hidden layers (326 neurons in each layer) with L₁ penalty of 6.0E-5 and L₂ penalty of 1.6E-4 will be constructed. A SVM model combining DL-known and DL-others will be then trained in the training cohort combining the DL-known and DL-others models.

A significant improvement in prediction performance will be observed using the deep learning algorithm compared to LDpred. In the test set, the Area Under the Curve (AUC) of the LDpred approach (with p-value cutoff of 0.01) will be about 0.7, while deep learning constructed using the known variants and variants in LD with known (DL-known) will exhibit an AUC of about 0.8, which will be significantly higher than that of LDpred. Deep learning with other variants (DL-others), where variants included in the DL-known analysis that will be excluded, will exhibit an AUC of about 0.7, which will be also higher than the LDpred prediction. Combining the DL-known and DL-other variants (DL-comb) will improve the overall AUC of prediction to about 0.8, which is among the best performance of risk prediction of complex human diseases using genetic data.

This improvement in prediction accuracy will lead to greatly enriched cases in both the extreme and even the not-so-extreme tails of the DL-comb scores. All deep-learning based approaches, whether based on the known variants, the remaining of the immunoChip, or the combined, will demonstrate better performance as compared to LDpred. For example, in the top 5% of the predicted risk, an Odds Ratio (OR) of about 10 will be observed for DL-others, an OR of about 20 to 25 will be observed for DL-known, and an OR of about 25 to 30 will be observed for DL-comb, compared to the rest of the 95% of the samples (as compared to about 6 for LDpred). As another example, in the top 10% of the predicted risk, observed Odds Ratios (OR) will be about 8 for DL-others, about 15-20 for DL-known, and about 20 for DL-comb, compared to about 4 for LDpred. Within the top 5% and 10% of the DL-comb score, about 90% will be scleroderma patients. As a comparison, in LDPred algorithm proportion of scleroderma patients will be about 80% and about 70%, respectively in the top 5% and 10%. The corresponding positive likelihood ratio (LR+) will be about 3 for DL-comb using top 5% and 10% cutoff, and about 2 for LDpred. And the corresponding negative likelihood ratio (LR−) will be about 0.1 to 0.2 for DL-comb, and about 0.3 to 0.5 for LDPred, respectively. These all indicate that the deep learning algorithms may greatly boost the practical potentials of genomic prediction.

Similar performance of the DL algorithm will be observed in additional cohorts, thereby confirming the DL model's robustness in independent and heterogenous test cohorts.

The variance importance metrics of the DL-other model in scleroderma prediction will be examined. The variable importance metrics from the DL algorithm indicates the relative importance or contribution of each variable and/or mutation to the overall model, which may be helpful in discovery of novel signals. For the top 500 variants, a meta-analysis will be performed incorporating the immunoChip data from the European Scleroderma Group, the Australia Scleroderma Group, and two US SSc cohorts. Of those 500 variants, it is expected that about 10 or more novel genome-wide signals will be identified with a meta-analysis p-value of about 1×10⁻⁹.

Conclusion: With this DL model, individuals with disease risk for a fibrotic disease, such as scleroderma, may be identified using only genetic data, making it a powerful tool for disease early diagnosis and intervention. The utility of the DL model is not limited to predicting complex disease risk for scleroderma. Without being bound by any particular theory, this DL model may be utilized with a wide range of genetic data input (e.g., genetic variants associated with other fibrotic diseases) to predict complex disease risk.

### Example 9: Using DeepLearning and Genetic BigData to Construct a Disease Prediction Model for Fibrosis in Pulmonary Fibrosis

Using systems and methods of the present disclosure, a Deep Learning (DL) model is built, validated and tested to predict fibrosis in subjects suffering from pulmonary fibrosis using genetic data. The performance of the DL model in this example is compared to the performance of LDpred. The DL model in this example and according to various embodiments described herein will yield more accurate predictions as compared to LDpred, underscoring the clinical utility of the DL model in clinical practice to inform decision-making (e.g., diagnosis, prognosis, selection of therapeutic intervention, disease and/or therapeutic regimen monitoring, and the like).

Methods: DL is utilized to build a disease prediction model with cases and controls selected from IPF case-control collections and UUS cohorts. 3,668 pulmonary fibrosis cases and 2,874 non-pulmonary fibrosis controls are selected from the IPF case-control collections, as the training dataset. This model will be further validated in 456 pulmonary fibrosis cases and 2,874 non-pulmonary fibrosis controls from the UUS cohorts, that are independent from the training set. Both training and validation cohorts are genotyped using ImmunoChip. A set of 10.3 million variants that are successfully measured and passed the stringent QC are included as predictors after imputation. A convolutional neural network (CNN) algorithm is used to construct a DL model, and cross-validation is performed as part of the DL model construction. Further, the association of the DL prediction score is examined with clinical phenotypes.

Performance of the DL model is compared to the LDpred algorithm (e.g., as described by Amit V. Khera et. al, Nature Genetics, 2018; which is incorporated herein by reference in its entirety). A non-trivial improvement in prediction performance of DL is observed with an Area Under the Curve (AUC) of about 0.8, as compared to about 0.7 using LDPred. The predicted risk from DL will lead to greatly enriched cases in the extreme of the DL score, with an OR of 15 with a P=1E-10 in the validation cohort. Utilizing only a set of about 500 known disease-susceptibility variants (and variants in LD with known), the DL based algorithm (DL-known) will achieve an AUC of about 0.8. Further analyses will indicate that the improved performance of the DL-known score is likely through its ability to incorporate non-linear causal effects. Moreover, after excluding known variants, an AUC of about 0.7 will be observed with the DL algorithm (DL-other). Variance importance metrics of the DL-other algorithm will identify novel variants that will achieve a genome-wide significance in a meta-analysis incorporating a large set of over 100,000 individuals. Via DL analysis, the expected predicted risk score will be also strongly associated with pulmonary fibrosis clinical phenotypes including disease location, severity, and need for therapeutic interventions. Therefore, utilizing this genetic algorithm, individuals with disease risk for pulmonary fibrosis will be identified, a capability that provides progress towards early diagnosis and identifying subjects for studying preventative strategies.

Subjects will be enrolled as follows. A training cohort will be obtained, comprising individuals from the IPF case-control collections. Subject recruitment will include recruiting a set of 3,668 pulmonary fibrosis cases and 2,874 non-pulmonary fibrosis controls. For each of these subjects, a diagnosis of pulmonary fibrosis will be made based on accepted clinical evaluation (e.g., radiological, endoscopic, and histopathological evaluation). All included cases will fulfill clinical criteria for pulmonary fibrosis and provided written consent. The entire cohort (the IPF case-control collections and UUS cohorts), after excluding any overlap with a test cohort, will be used as a training dataset.

An independent cohort from the UUS cohorts will be used as a test cohort to generate a test dataset. Validation will be performed in this test cohort of 456 pulmonary fibrosis cases and 2,874 non-pulmonary fibrosis controls. The patient recruitment for the UUS cohorts will include pulmonary fibrosis cases and non-pulmonary fibrosis control cases with genotype data (after QC). For each of these subjects, a diagnosis of pulmonary fibrosis will be made based on standard clinical features (e.g., endoscopic, histologic, and radiographic features). The study protocol and data collection, including DNA preparation and genotyping, will be approved by the Institutional Review Board. Written informed consents will be obtained from all study participants.

Genotyping and genotype quality control (QC) will be performed as follows. All cohorts will be genotyped using Illumina ImmunoChip™ platform. Further, QC in the cohorts will be performed. In brief, the ImmunoChip™ samples will be genotyped in 36 batches, and genotype calling will be performed separately for each batch. Stringent QC will be performed, removing the following: SNPs with a call rate lower than 98% across all genotyping batches or 90% in one of the genotyping batches, SNPs that do not appear in the 1000 Genomes Project Phase I, SNPs that failed Hardy-Weinberg Equilibrium (P<10⁻⁵ across all samples or within each genotyping batch), and monomorphic SNPs. Individuals will be assigned to different populations based on principal components, and those not in the European Ancestry cluster, and those with low call rate (less than 98%), outlying heterozygosity rate (P<0.01) or cryptic relatedness (identity by decent >0.4) will be removed.

Genotyping of the validation cohort will be performed using an Illumina ImmunoChip™ array. Individual and genotype missingness, allele frequencies, and deviations from Hardy-Weinberg Equilibrium will be calculated using the PLINK software package (pngu.mgh.harvard.edu/˜purcell/plink). Individual-level QC thresholds will be used, including a genotyping call rate of greater than 95% and an inbreeding coefficient of less than 0.05. Ethnicity outliers that are identified using Admixture software will also be removed. SNPs with a call rate of less than 0.95, minor allele frequency (MAF) of less than 0.01, and strong deviation from Hardy-Weinberg equilibrium (P<10⁻⁷) will also be removed.

A set of 10.3 million SNPs available post-QC in the IPF case-control collections and UUS cohorts, will be selected for further analyses. Of these, about 500 are known pulmonary fibrosis variants or in LD with known pulmonary fibrosis variants with r2>0.2 in 1000 Genome Project Phase3 data.

The Deep Learning prediction models will be constructed as follows. A multi-layer feedforward artificial neural network, also known as convolutional neural network (CNN), will be utilized to build the prediction model. The CNN model will be constructed separately with a) the about 500 variants that are either known or in LD (r2>0.2) with known pulmonary fibrosis variants (DL-known), and b) the remaining variants not in LD with known variants (DL-others). The CNN model will be optimized in the software H2O using stochastic gradient descent with both L₁ and L₂ regularization. A grid search will be performed to determine the best parameter settings separately for DL-known and DL-others, including numbers of hidden layers, number of neurons in each layer, activation functions of the layers, dropout ratio, and parameters for L₁ and L₂ regularization. In the trained models, the variable relative importance will be calculated using Gedeon's approach, based on the weights connecting the input features to the first two hidden layers. A 5-fold cross-validation will be applied to the control for model overfitting. Further, an ensemble model (DL-comb) based on Support Vector Machine (SVM) will be built to combine DL-known and DL-others with 5-fold cross-validation. After building up different Deep Learning models in the training dataset, models are fitted using the test datasets. The final prediction model will be incorporated into a pulmonary fibrosis risk prediction tool.

Prediction performance of the deep learning algorithm will be compared to the LDPred approach as follows.

LDpred analysis will be performed using the default parameters, based on the public available summary statistics. The LDPred23 Python package will be used for these analyses. LDPred analysis will be performed across different p-value thresholds (1.0E-6, 3.0E-6, 1.0E-5, 3.0E-5, 1.0E-4, 3.0E-4, 0.001, 0.003, 0.01, 0.05, 0.10, and 0.25), and the p-value threshold with best AUC will be selected.

Prediction performance will be evaluated as follows. Receiver Operating Characteristic (ROC) curves will be generated for different prediction models in the test dataset. Further, Area Under Curve (AUC) will be calculated for each of the ROC curves, and compared using the R package pROC31. Further, the performance of difference approaches will be evaluated in enrichment of pulmonary fibrosis cases in the extreme of pulmonary fibrosis risk prediction. All comparisons will be performed in the R software package.

High-order combination analysis will be performed as follows. To investigate the effects of non-linear effects in known variants (and variants associated with known), the combined effects of variants used in DL-known analysis will be examined using LAMPlink software. Combinations of both dominant and recessive models are performed, and LD filtering will be performed with r2 cutoff of 0.2 to exclude potential contamination from SNPs in strong LD.

Association of single variants with pulmonary fibrosis and meta-analysis will be performed as follows. Association of SNPs within the top 500 of variable importance with pulmonary fibrosis will be examined in the IPF case-control collections and UUS cohorts separately, using logistic regression with adjustment for principal components from population stratification analysis. A meta-analysis will be performed to combine the summary statistics in both cohorts, after excluding overlapping samples.

Association of predicted risk with pulmonary fibrosis clinical phenotypes will be performed as follows. Association of prediction score from different algorithms with clinical characteristics will be evaluated in the generalized linear model framework, with Principal Components from population stratification analysis included as covariates.

Results

After performing an intensive grid search to tune or optimize the hyperparameters, a CNN model with three hidden layers (154 neurons in each layer, with L₁ penalty of 5.0E-5 and L₃ penalty of 1.0E-4) will be constructed for DL-known. For DL-others, a model with two hidden layers (326 neurons in each layer) with L₁ penalty of 6.0E-5 and L₂ penalty of 1.6E-4 will be constructed. A SVM model combining DL-known and DL-others will be then trained in the training cohort combining the DL-known and DL-others models.

A significant improvement in prediction performance will be observed using the deep learning algorithm compared to LDpred. In the test set, the Area Under the Curve (AUC) of the LDpred approach (with p-value cutoff of 0.01) will be about 0.7, while deep learning constructed using the known variants and variants in LD with known (DL-known) will exhibit an AUC of about 0.8, which will be significantly higher than that of LDpred. Deep learning with other variants (DL-others), where variants included in the DL-known analysis that will be excluded, will exhibit an AUC of about 0.7, which will be also higher than the LDpred prediction. Combining the DL-known and DL-other variants (DL-comb) will improve the overall AUC of prediction to about 0.8, which is among the best performance of risk prediction of complex human diseases using genetic data.

This improvement in prediction accuracy will lead to greatly enriched cases in both the extreme and even the not-so-extreme tails of the DL-comb scores. All deep-learning based approaches, whether based on the known variants, the remaining of the immunoChip, or the combined, will demonstrate better performance as compared to LDpred. For example, in the top 5% of the predicted risk, an Odds Ratio (OR) of about 10 will be observed for DL-others, an OR of about 20 to 25 will be observed for DL-known, and an OR of about 25 to 30 will be observed for DL-comb, compared to the rest of the 95% of the samples (as compared to about 6 for LDpred). As another example, in the top 10% of the predicted risk, observed Odds Ratios (OR) will be about 8 for DL-others, about 15-20 for DL-known, and about 20 for DL-comb, compared to about 4 for LDpred. Within the top 5% and 10% of the DL-comb score, about 90% will be pulmonary fibrosis patients. As a comparison, in LDPred algorithm proportion of pulmonary fibrosis patients will be about 80% and about 70%, respectively in the top 5% and 10%. The corresponding positive likelihood ratio (LR+) will be about 3 for DL-comb using top 5% and 10% cutoff, and about 2 for LDpred. And the corresponding negative likelihood ratio (LR−) will be about 0.1 to 0.2 for DL-comb, and about 0.3 to 0.5 for LDPred, respectively. These all indicate that the deep learning algorithms may greatly boost the practical potentials of genomic prediction.

Similar performance of the DL algorithm will be observed in additional cohorts, thereby confirming the DL model's robustness in independent and heterogenous test cohorts.

The variance importance metrics of the DL-other model in pulmonary fibrosis prediction will be examined. The variable importance metrics from the DL algorithm indicates the relative importance or contribution of each variable and/or mutation to the overall model, which may be helpful in discovery of novel signals. For the top 500 variants, a meta-analysis will be performed incorporating the immunoChip data from the IPF case-control collections and UUS cohorts. Of those 500 variants, it is expected that about 10 or more novel genome-wide signals will be identified with a meta-analysis p-value of about 1×10⁻⁹.

Conclusion: With this DL model, individuals with disease risk for a fibrotic disease, such as pulmonary fibrosis, may be identified using only genetic data, making it a powerful tool for disease early diagnosis and intervention. The utility of the DL model is not limited to predicting complex disease risk for pulmonary fibrosis. Without being bound by any particular theory, this DL model may be utilized with a wide range of genetic data input (e.g., genetic variants associated with other fibrotic diseases) to predict complex disease risk.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for identifying a fibrotic disease or condition in a subject, comprising:
(a) assaying a biological sample of the subject to generate a dataset comprising genetic data;
(b) processing the dataset at a plurality of genomic loci to determine quantitative measures of each genomic locus of the plurality of genomic loci, wherein the plurality of genomic loci comprises fibrotic disease-associated genes, thereby producing fibrotic disease profile of the biological sample of the subject; and
(c) applying a deep learning prediction model to the fibrotic disease profile to identify a presence or an absence of the fibrotic disease in the subject, or a likelihood that the subject will develop the fibrotic disease.

2. The method of claim 1, wherein the fibrotic disease comprises Primary Sclerosing Cholangitis (PSC), scleroderma, or pulmonary fibrosis.

3. The method of claim 2, wherein the fibrotic disease comprises the PSC.

4. The method of claim 2, wherein the fibrotic disease comprises the scleroderma.

5. The method of claim 2, wherein the fibrotic disease comprises the pulmonary fibrosis.

6. The method of claim 1, wherein the biological sample is selected from the group consisting of: a whole blood sample, a DNA sample, an RNA sample, a cell-free sample, a tissue sample, a cell sample, and a derivative or fraction thereof.

7. The method of claim 1, wherein assaying the biological sample comprises sequencing the biological sample to generate the dataset.

8. The method of claim 1, further comprising identifying the presence or the absence of the fibrotic disease in the subject, or the likelihood that the subject will develop the fibrotic disease, at a sensitivity of at least about 70%, at least about 80%, or at least about 90%.

9. The method of claim 1, further comprising identifying the presence or the absence of the fibrotic disease in the subject, or the likelihood that the subject will develop the fibrotic disease, at a specificity of at least about 70%, at least about 80%, or at least about 90%.

10. The method of claim 1, further comprising identifying the presence or the absence of the fibrotic disease in the subject, or the likelihood that the subject will develop the fibrotic disease, at a positive predictive value of at least about 70%, at least about 80%, or at least about 90%.

11. The method of claim 1, further comprising identifying the presence or the absence of the fibrotic disease in the subject, or the likelihood that the subject will develop the fibrotic disease, at a negative predictive value of at least about 70%, at least about 80%, or at least about 90%.

12. The method of claim 1, further comprising identifying the presence or the absence of the fibrotic disease in the subject, or the likelihood that the subject will develop the fibrotic disease, with an Area Under Curve of at least about 0.70, at least about 0.80, or at least about 0.90.

13. The method of claim 3, wherein the subject is asymptomatic for the fibrotic disease.

14. The method of claim 1, wherein the deep learning prediction model is trained using a first set of independent training samples associated with a presence of the fibrotic disease and a second set of independent training samples associated with an absence of the fibrotic disease.

15. The method of claim 1, further comprising applying the deep learning prediction model to a set of clinical health data of the subject.

16. The method of claim 1, wherein the deep learning prediction model comprises a deep learning algorithm, a neural network, a Random Forest, an XGBoost, a Gradient Boost, or a combination thereof.

17. The method of claim 16, wherein the deep learning prediction model comprises a deep learning algorithm.

18. The method of claim 17, wherein the deep learning algorithm comprises a deep neural network.

19. The method of claim 18, wherein the deep neural network comprises a convolutional neural network (CNN).

20. The method of claim 19, further comprising optimizing a set of hyperparameters of the CNN.

21. The method of claim 20, wherein optimizing the set of hyperparameters comprises performing an intensive grid search.

22. The method of claim 20, wherein the set of hyperparameters comprises a number of layers and/or a number of neurons of the CNN.

23. The method of claim 1, wherein (a) comprises (i) subjecting the biological sample to conditions that are sufficient to isolate, enrich, or extract a plurality of DNA molecules; and (ii) analyzing the plurality of DNA molecules to generate the dataset.

24. The method of claim 1, wherein the plurality of genomic loci comprises at least about 1,000 distinct genomic loci, at least about 10,000 distinct genomic loci, or at least about 100,000 distinct genomic loci.

25. The method of claim 1, further comprising identifying the likelihood that the subject will develop the fibrotic disease.

26. The method of claim 1, further comprising providing a therapeutic intervention for the fibrotic disease of the subject, provided the presence of the fibrotic disease is identified in the subject.

27. The method of claim 1, further comprising monitoring the fibrotic disease of the subject by assessing the fibrotic disease in the subject at a plurality of time points, wherein the assessing is based at least partially on identifying the presence of the fibrotic disease in (c) at one or more time points of the plurality of time points.

28. The method of claim 29, wherein a difference between two or more assessments of the fibrotic disease in the subject at two or more time points of the plurality of time points is indicative of one or more of: (i) a diagnosis of the fibrotic disease of the subject, (ii) a prognosis of the fibrotic disease of the subject, or (iii) an efficacy or non-efficacy of a course of treatment for treating the fibrotic disease of the subject.

29. A computer system for identifying a fibrotic disease of a subject, comprising:
(a) a database that is configured to store a dataset comprising genetic data, wherein the genetic data is obtained by assaying a biological sample of the subject; and
(b) one or more computer processors operatively coupled to the database, wherein the one or more computer processors are individually or collectively programmed to:
(i) process the dataset at a plurality of genomic loci to determine quantitative measures of each genomic locus of the plurality of genomic loci, wherein the plurality of genomic loci comprises fibrotic disease-associated genes, thereby producing a fibrotic disease profile of the biological sample of the subject; and
(ii) apply a deep learning prediction model to the fibrotic disease profile to identify a presence or an absence of the fibrotic disease in the subject, or a likelihood that the subject will develop the fibrotic disease.

30. A non-transitory computer-readable medium comprising machine-executable code that, upon execution by one or more computer processors, implements the method of claim 1.
